(19) United States
(12) Patent Application Publication
(10) Pub. No.: US 2008/0138828 A1
(43) Pub. Date: Jun. 12, 2008

(54) Microgravity bioreactor systems for production of bioactive compounds and biological macromolecules

(75) Inventors: Jagan V. Valluri, Huntington, WV (US); Steven R. Gonda, Houston, TX (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Marshall University Research Corporation

(21) Appl. No.: 11/635,785

(22) Filed: Dec. 8, 2006

(51) Int. Cl. C12N 1/20; C12N 1/00; C12N 5/00; C12P 21/00; G01N 33/53

(52) U.S. Cl. 435/7.1; 435/253.6; 435/256.8; 435/41; 435/420

(57) ABSTRACT

The present invention relates to compositions and methods of plant, fungal and bacterial cell culture that can be effectively utilized for three-dimensional plant, fungal, and bacterial cell growth and production of bioactive compounds of interest. The culture methods employ a microgravity environment such that rapid establishment and expansion of cells into tissue constructs occurs influencing expression of biological macromolecules and biopharmaceuticals. The present invention is further directed to a method for the in vitro cultivation of plant, fungal, and bacterial cells in a liquid nutrient medium in modeled microgravity with potential for large scale manipulations.

### RESEARCH AND DEVELOPMENT

Statement under MPEP 310. The U.S. government has a paid-up license in this invention and the right in limited circumstances to require the patent owner to license others on reasonable terms as provided for by the terms of NTG5-40120 awarded by the National Aeronautics and Space Administration (NASA).

Part of the work performed during development of this invention utilized U.S. Government finds. The U.S. Government has certain rights in this invention.

### BACKGROUND OF THE INVENTION

1. Field of the Invention

The methods of the present invention relate to a three-dimensional cell culture process. Through the present invention, plant, fungal and bacterial cells are cultured in microgravity to produce tissue-like, three-dimensional cell constructs which have the ability to express bioactive compounds of interest.

2. Background Art

Plant cells are important biocatalysts that can be used for the production of a wide range of bioactive compounds including pharmaceuticals (codeine, scopalamine, vincristine, ajmalicine, and digoxin); flavors and fragrances (strawberry, vanilla, rose, and lemon); sweeteners (thaumatin and monellin); food colors (anthocyanin and saffron) and pesticides (thiophenes, azadirachtins, nicotine). Large markets exist for these bioactive compounds, which are normally obtained by extraction from intact plants. In view of the growing world population, increasing anthropogenic activities and rapidly eroding natural ecosystems, the natural habitats for a large number of plants are rapidly dwindling leading to the extinction of many valuable species.

A number of plants such as Catharanthus roseus (vincristine, vinblastine, ajmalicine), Taxus baccata (taxol), Nothapodytes foetida (camptothecin), and Artemisia annua (artemisinin) have been screened for anti-cancer, anti-AIDS, anti-malarial and other useful bioactive compounds for therapeutic use (Zhong, J. J., Adv. Biochem. Eng. Biotechnol. 72:1-26 (2001)). The Apocynaceae plant family, which contains the important medicinal plant Madagascar Periwinkle (Catharanthus roseus G. Don), is characterized by the large variety of monoterpenoid indole alkaloids that it produces. The alkaloid chemistry of many members of this family has been well characterized and several thousand structures have been elucidated. Among these many structures, vinblastine and vincristine from Madagascar Periwinkle are of particular importance because of their wide use in cancer chemotherapy. These alkaloids are produced in vivo by the condensation of vinoline and catharanthine. The pharmaceutical value of these dimeric alkaloids, their low abundance, and their cost of production has prompted extensive efforts to generate cost efficient high-yielding cell and organ cultures of Madagascar Periwinkle.

Medicinal plants are the most exclusive source of life saving drugs for the majority of the world's population. More than 80% of the world's population continues to depend on plants for their medicinal needs (Farnsworth, N. R. “Screening plants for new medicines,” in Biodiversity, Wilson, E. O., ed., National Academy Press, Washington, D.C. (1988)). Further, it has been reported that 37% of the 100 most prescribed drugs contain one or more active ingredients of plant origin (Farnsworth, N. R., “The role of ethnopharmacology in drug development,” in Bioactive Compounds from Plants. Ciba Foundation Symposium 154, Chadwick, D. J., and J. Marsh, eds., John Wiley and Sons, New York. (1990)).

Older and well-established examples of plant drugs include morphine, quinine, cocaine and digitalis. More recently, bioprospecting by the National Cancer Institute (NCI) has produced an array of compounds with potential to treat cancer and HIV (Cragg, G. M., Ann. Missouri Bot. Gard. 82:47-53 (1995)). NCI efforts have demonstrated the value of focusing on plants used in traditional medicine versus plants selected at random. Preliminary testing showed bioactive compounds in 25% of plants with a history of use in traditional medicine versus 6% in plants chosen at random (Balick, M. J., “Ethnobotany and the identification of therapeutic agents from the rain forest,” in Bioactive Compounds from Plants. Ciba Foundation Symposium 154, Chadwick, D. J, and Marsh, J., eds, John Wiley and Sons, New York (1990)). Some of the most recent and spectacular finds include taxol from the Pacific Yew, uncommonly effective in treating breast and lung cancer; vincristine and vinblastine, alkaloids from the Periwinkle plant, of prime importance in treating childhood leukemia and Hodgkin's Disease (Cragg, G. M., Ann. Missouri Bot. Gard. 82:47-53 (1995)). Successes such as these have led to increased testing of plants used in traditional medicine (Villacres O., V. H., Bioactividad de plantas amazonicas, Abya-Yala Press, Quito, Ecuador (1995); Bruneton, J., Pharmacognosy, Phytochemistry, and Medicinal Plants, Lavoisier Publishing, Paris (1995), 915 pp.).

Another area of active research includes the production of “plant-made pharmaceuticals” (PMPs). PMPs are produced by genetically engineering plant cells to produce specific compounds, generally proteins, which are extracted and purified after harvest. These pharmaceuticals promise more plentiful and cheaper supplies of pharmaceutical drugs, including vaccines for infectious diseases and therapeutic proteins for the treatment of cancer and heart disease.

Many drugs derived from natural products have yet to be synthesized in the laboratory and thus supply remains based upon crude plant materials. One alternative to field grown plants is to culture plant cells in bioreactors under controlled defined parameters, while retaining the biosynthetic capacity to synthesize bioactive compounds. Unlike field grown plants, bioreactor-grown plant cell cultures may prove an excellent source of bioactive compounds because these cell cultures do not suffer from diseases, pests and climatic restraints. (See e.g., Collin, H. A., Plant Growth Regulation 34:119-134 (2001).) Bioreactor applications of plant cell cultures would also allow isolation of an unlimited supply of biologically active compounds. Bioreactors would provide a closely controlled environment for the optimum growth of plant cells in which cells perform biochemical transformation to synthesize bioactive compounds. Bioreactors have several advantages over traditional cell cultures for the mass cultivation of cells. They provide better control for scale up of cell suspension cultures under defined parameters for the production of bioactive compounds. Constant regulation of conditions at various stages of bioreactor operation is possible. Handling of culture such as inoculation or harvest is easy and saves time. Nutrient uptake is enhanced by submerged culture conditions which stimulate cell multiplication rate and promote higher yield of bioactive compounds.

Bioreactors can also be used to culture plant cells to provide food and replenished air supplies for the spacecraft, or planetary colony (Blum, V., et al., “Novel laboratory approaches to multi-purpose aquatic biogenerative closed-loop food production systems,” in Proceedings of the 12th Man in Space Symposium, June 8-13, Washington, D.C. (1997), pp. 17-18; Gitelson, J. I., et al., Acta Astronautica 37:385-394 (1995); Klymchuk, D. O., Journal of Gravitational Physiology 5:147-148 (1998)).

Production of shikonin in bioreactors by cell cultures of Lithospermum erythrorhizon plant cells was the first instance of a commercial large-scale process using plant cell suspensions (Fujita, Y., et al., “New medium and production of secondary compounds with the two stage culture method,” in: Proc. 5th Intl. Cong. Plant Tissue and Cell Cultures, Fujiwara, ed., Maruzen Co., Tokyo (1992), pp 399-400). Medicinal plants such as Sandalwood (Santalum album L.), Periwinkle (Catharanthus roseus), and Kantikari (Solanum Xanthocarpum) are plant species whose cells could be cultured in bioreactors (Valluri, J. V., et al., Plant Cell Rep 10:366-370 (1991); Valluri J. V., “Santalum album L. (Sandalwood): In Vitro culture and the bioreactor production of secondary metabolites, “in: Biotechnology in Agriculture and Forestry 28, Medicinal and Aromatic Plants VII, Bajaj YPS, ed., Springer, Berlin Heidelberg New York (1994), pp 401-411).

Plant cells in liquid suspensions offer a unique combination of physical and biological properties that must be accommodated in large-scale bioreactor processes aimed at exploiting their biomass and synthesis of bioactive compounds. Plant cells have rigid cell walls and tend to grow very slowly with doubling times of days rather than hours. Cultured plant cells range from 30-100 μm in diameter and are 10 to 100 times larger than bacterial and fungal cells. They contain vacuoles occupying 95% or more of the cell's volume and are destroyed by impeller speeds as low as 28 RPM. Plant cell suspensions tend to stick to fermenter surfaces and become very thick as they grow.

This adhesive characteristic combined with the shear sensitivity means it is often difficult to attain good oxygen transfer with conventional bioreactor culture. Suspension of cells is easily achievable using stirred technologies. Unfortunately, in impeller-driven bioreactors stirring invokes deleterious forces that disrupt cell aggregation and results in cell death.

The hydrodynamic environment of the stirred-tank bioreactor, in which plant cells are sensitive to fluid forces and gas composition, makes the tasks of producing three-dimensional growth and tissue differentiation difficult. (Payne, G. F., et al., in Plant Cell and Tissue Culture in Liquid Systems, Hanser Publishers, Munich (1991), Chapters 1 and 6; Taticek, R. A., et al., Plant Cell, Tissue and Organ Culture 24:139-158 (1991)).

Furthermore, the requirements for media oxygenation create a foaming in the bioreactor, which also tends to perturb and otherwise damage cells. These factors limit the concentration and density of the bioreactor nutrient culture medium. The conventional bioreactor approach for growing plants has the disadvantage that the mechanically stirred impellers, which damage cells, generate high shear forces and hinder proper tissue-specific differentiation.

The NASA first generation rotating bioreactors provided rotation about the horizontal axis which resulted in the suspension of cells without stirring, thus providing a suitable environment to propagate cells without sedimentation to a surface. Unfortunately, these first generation High Aspect Rotating Vessel (HARV) bioreactors do not provide a way to remove air bubbles that are disruptive to the survival of plant cells and the integrity of the tissue-like, three-dimensional plant cell constructs. When the HARV bioreactor is used, the cell growth rate is very slow compared to the general shake-flask culture method, because the lag phase is longer in order to fit the circumstance of microgravity.

Therefore, there is a need for improved techniques for culturing tissue-like, three-dimensional plant cell constructs in a low-shear cell culture environment to increase the survival and maintain the integrity of three-dimensional plant tissues. Cell aggregation or artificial confinement in a porous support is frequently either necessary or desirable to stimulate certain secondary metabolite pathways. Excellent control over cell residence time is desirable so that the cells can be induced at the appropriate time for product formation.

### BRIEF SUMMARY OF THE INVENTION

In the present invention, a hydrofocusing bioreactor (HFB) is used to culture and grow plant, fungal and bacterial cells and tissue-like, three-dimensional cell constructs. The three-dimensional cell tissues grown in the hydrofocusing bioreactor provide an excellent in vitro system for studying the micro-environmental cues on tissue-specific cell assembly, differentiation and function.

One of the many embodiments of the present invention is directed to a method for continuous culture of plant cells by growing the cells in a hydrofocusing bioreactor.

An aspect of the present invention is directed to a method for producing one or more bioactive compounds, by continuously culturing plant cells in a hydrofocusing bioreactor.

Another aspect of the present invention is directed to a method for increasing the production of one or more bioactive compounds in plant cells cultured in a hydrofocusing bioreactor compared to levels of bioactive compounds in plant cells cultured in shake-flasks.

Another embodiment of the present invention is directed to a method for increasing the production of one or more bioactive compounds in induced plant cells cultured in a hydrofocusing bioreactor compared to levels of bioactive compounds in uninduced plant cells cultured in a hydrofocusing bioreactor.

Another aspect of the present invention is directed to a method for assaying the presence of one or more bioactive compounds by continuously culturing plant cells in a hydrofocusing bioreactor.

Another aspect of the present invention is directed to a process of producing tissue-like, three-dimensional plant cell constructs.

Another aspect of the present invention is directed to a method for continuous culture of anchorage-dependent plant cells by growing the cells in a hydrofocusing bioreactor with media containing attachment material such as microcarrier beads.

Another aspect of the present invention is directed to a tissue-like, three-dimensional plant cell construct grown in a hydrofocusing bioreactor.

Another aspect of the present invention is directed to a method for continuous culture of fungal cells by growing the cells in a hydrofocusing bioreactor.

Another aspect of the present invention is directed to a method for continuous culture of bacterial cells by growing the cells in a hydrofocusing bioreactor.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 shows a hydrofocusing bioreactor with a 160 mL culture chamber. At the apex of the dome-shaped culture chamber is a sampling port.

FIG. 2 shows a laser confocal microscopy image of Periwinkle plant cells that have been harvested at the end of 7 days of culture in the HFB. Plant chloroplasts are easily distinguishable as ˜3 μM particles that are present within the plant cells. The micrographs show that there are no observable disturbances in chloroplast structure in plant cells subjected to microgravity in the HFB, compared to chloroplasts in plant cells grown in shake-flasks.

FIG. 3 is a laser confocal microscopy image of Periwinkle plant cells that have been harvested at the end of 7 days of culture in the HFB. Plant chloroplasts are easily visualized in this micrograph. Compared to plant cells that have not been cultured in the HFB, the chloroplasts in HFB culture plant cells exhibit swelling.

FIG. 4 shows the formation of tissue-like, three-dimensional Periwinkle plant cell constructs at the end of 3 days of culture. The three-dimensional plant tissues are clearly evident within the bioreactor culture chamber and are maintained in a state of microgravity.

FIG. 5 shows the formation of tissue-like, three-dimensional Periwinkle plant cell constructs at the end of 4 days of culture. Again, the three-dimensional plant tissues are clearly evident within the bioreactor culture chamber and are maintained in a state of microgravity.

FIG. 6 shows the F-actin cytoskeleton of Periwinkle plant cells that are not grown under microgravity conditions. Cells were harvested at the end of a 7 day culture in shake-flasks. The laser confocal microscopy image illustrates that under normal gravity conditions, the F-actin cytoskeleton inside plant cells is dense and exhibits a meshlike network of filaments.

FIG. 7 shows the F-actin cytoskeleton in Periwinkle plant cells that were grown under microgravity conditions. Cells were harvested at the end of a 7 day culture in the HFB. The laser confocal microscopy image illustrates that under microgravity conditions, the F-actin cytoskeleton of plant cells reorganizes and degrades following exposure to altered environmental conditions.

FIG. 8 shows the Sodium Dodecyl Sulfate Polyacrylamide gel electrophoresis (SDS-PAGE) protein expression profiles from Periwinkle plant cells cultured over a 7 day period in the HFB. The expression levels of various proteins produced within the plant cells varies over the culture time in the HFB. An 85 kD protein is expressed in greater quantities in the HFB grown cells compared to the protein expression levels from control cells. There is also a partial enhancement in the expression of a 43 kD protein followed by decline in that protein's expression over the 7 day time period.

FIG. 9 shows G-actin immunoblots from Periwinkle plant cells after the actin protein has been submitted to 2-D SDS-PAGE electrophoresis. The molecular mass of the immunostained spots is 43 kDa, which is the molecular weight of G-actin. Periwinkle plants have four actin isoforms, which are constitutive polypeptides and show a distinct distribution within the specific cellular compartments: two isoforms (pI 5.9 and 6.0) were found in the plasma membrane and tonoplast preparations, whereas the pI 5.95 and 6.05 polypeptides were present in the soluble fraction. Arrowheads mark the different actin isoforms with pI values of (1) 6.05, (2) 6.0, (3) 5.95 and (4) 5.9. Panel 2A is a 2-D SDS-PAGE immunoblot from control cells cultured in a shake-flask. Panel 2B is a 2-D SDS-PAGE immunoblot from cells cultured in the HFB for 2 days and shows a slight increase in expression of the four major isoforms. Panel 2C is a 2-D SDS-PAGE immunoblot from cells cultured in the HFB for 5 days. Panel 2D is a 2-D SDS-PAGE immunoblot from cells cultured in the HFB for 7 days. The relative amounts of G-actin isoforms 1 and 3 (arrowheads) vary over culture time. The amount of G-actin isoform 1 that is being expressed in HFB cultured plant cells significantly decreases over culture time.

FIG. 10 shows isoflavonoid production by Sandalwood cells incubated over 80 hours. Control cells were grown for 80 hours in shake-flasks and samples for detecting isoflavonoids were withdrawn at 0, 4, 12, 20, 40, 60 and 80 hours. Sandalwood cells were induced using mannitol, an abiotic agent, and incubated over 80 hours in the HFB. Consistently over the time-course, the Sandalwood cells cultured in the HFB produced more isoflavonoids, in terms of mg/grams of dry weight of cells (gDW), than cells cultured in shake-flasks.

FIG. 11 shows the distribution of alkaloids produced when Periwinkle cells are osmotically stressed in an HFB culture. Mannitol was used to osmotically stress the Periwinkle cell cultures in the HFB. The mannitol does not allow water uptake by the plant cells, mimicking drought conditions. Although only present in μg/gDW, alkaloid production did occur within the Periwinkle cultures and showed a steady increase over a 7 hour period after mannitol induction. Alkaloid concentrations were measured in the medium as well as in the cell bodies. Alkaloid concentrations increased over time and were mainly present in the culture medium.

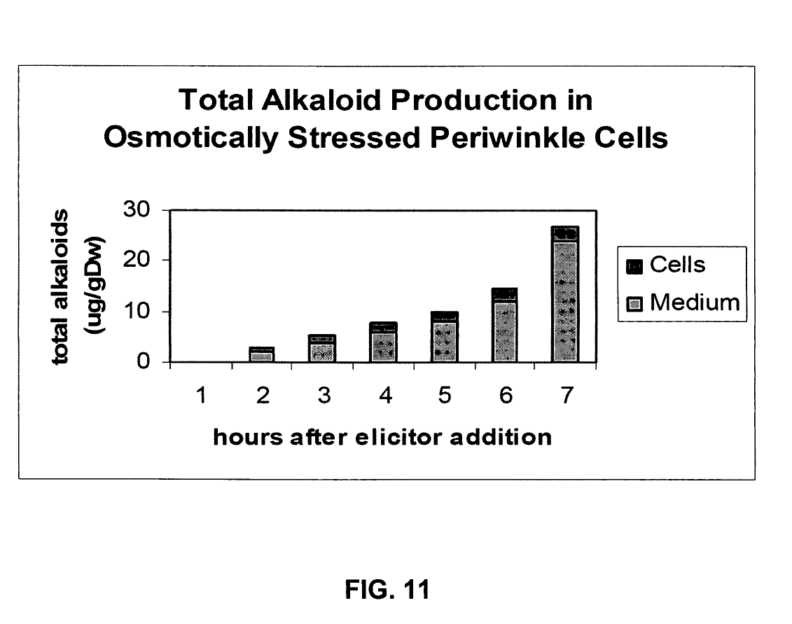

FIG. 12 shows the amount of alkaloids produced when Periwinkle cells were induced with a combination of inducing agents. The black diamonds represent the total alkaloid amounts produced by plant cells cultured in the absence of inducing agents over a 7 hour period in the HFB (control cells). The grey boxes represent the total alkaloid amounts produced by plant cells cultured in the presence of one inducing agent over a 7 hour period in the HFB. The inducing agent used was an Aspergillum niger mycelium extract. Over a 7 hour incubation period, the Periwinkle plant cells that had been induced with a biotic inducing agent, Aspergillum niger mycelium extract, produced more total alkaloids than the control cells. The grey triangles represent the total alkaloid amounts produced by plant cells cultured in the presence of two inducing agents over a 7 hour period in the HFB. The inducing agents used were a biotic inducing agent, Aspergillum niger mycelium extract, and an abiotic inducing agent, mannitol. The alkaloid production increased two and a half times when biotic and abiotic inducing agents were both added to the Periwinkle culture.

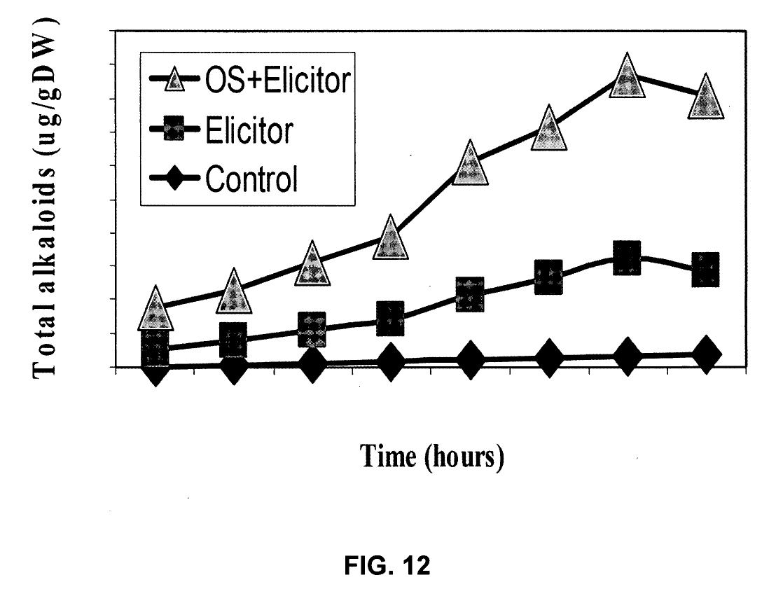

FIG. 13 shows the biomass of Escherichia coli cells grown in the HFB over a 20 hour incubation period. In panel A, optical density of culture medium is measured at 600 nm in 2 hour intervals. Optical density, an indicator of biomass, increases in the first 10 hours of incubation, after which it slowly decreases. In panel B, dry cell weight (DCW) is measured in mg/mL of culture medium in 2 hour intervals. DCW also increases in the first 10 hours of incubation, after which it slowly decreases.

### DETAILED DESCRIPTION OF THE INVENTION

The culture of plant cells in HFBs offers new opportunities for the metabolic engineering of plant cells. The HFB's simulation of microgravity offers a low shear environment, which promotes co-location of cells. Culture conditions in the HFB provide an excellent in vitro system for studying the microenvironmental cues especially intercellular communication on tissue-specific cell assembly, differentiation and function.

The Hydrodynamic Focusing Bioreactor (HFB) (see e.g., U.S. Pat. No. 6,001,642, which is hereby incorporated by reference) is a horizontally rotating, fluid-filled culture vessel equipped with a membrane for diffusion gas exchange to optimize gas/oxygen-supply capable of simulating microgravity. In the HFB, at any given time, gravitational vectors are randomized and the shear stress exerted by the fluid on the synchronously moving particles is minimized. These simulated microgravity conditions facilitate spatial co-location and three-dimensional assembly of individual cells into large tissues (Wolf, D. A, and Schwartz, R. P., Analysis of gravity-induced particle motion and fluid perfusion flow in the NASA-designed rotating zero-head-space tissue culture vessel, NASA Tech Paper 3143, Washington D.C. (1991)).

By the term “microgravity” is meant the near weightlessness condition created inside a spacecraft as it orbits the Earth. In the simulated microgravity environment of the HFB where there is no buoyancy, no convection, no stratification of layers, and where surface tension dominates, major impacts on metabolism will be reflected in the biosynthetic potential of cultured cells and protoplasts. There are also significant advantages of such a system over a 1-G microenvironment found in shaker flasks. For example, cell cultures can be grown and maintained under controlled conditions with respect to nutritional and environmental requirements. Such a situation would allow establishment of conditions for optimal cell growth or maximum bioactive compound formation, and for the selection of high producing genotypes; the cell culture methods would permit location of production facilities in any place without dependence on a region with certain anticipated or required climatic conditions; cultured cells would allow biochemical production to occur year-round in a reliable manner without interruptions due to agronomic practice, to season, or to other environmental factors or even political factors; biomass production by cells in rapidly growing cultures can be considerably more than in cells in situ; production in cell suspension culture should be automatable and this can lead to a significantly improved biotechnology; and provides the basis for disclosing principles which can lead to a still fuller understanding of the entire process of growth, metabolism, and differentiation.

Cell culture conditions in the simulated microgravity environment of the Hydrodynamic Focusing Bioreactor (HFB) combine two beneficial factors: low shear stress, which promotes the assembly of tissue-like, three-dimensional cell constructs; and randomized gravitational vectors, which affect the production of medicinal compounds. The shear sensitivity and rapid setting characteristics of plant cell tissues and the cell-floating tendencies of cell cultures can be overcome by using the Hydrodynamic Focusing Bioreactor (HFB). Close apposition of the cells in the absence of shear forces promotes cell-cell contacts, cell aggregation and cell differentiation. This process then leads to the rapid establishment and expansion of tissue-like cultures, which unlike cells cultured in conventional bioreactors, are not disrupted by shear forces.

This microgravity environment of the HFB keeps cells suspended in the fluid medium without imparting shear forces that are common in conventional bioreactors. Before the introduction of the HFB, the on-orbit formation of air bubbles in culture fluid and attempts at removing these bubbles from the fluid medium of the High Aspect Rotating Vessel (HARV) bioreactor degraded both the low-shear cell culture environment and the delicate three-dimensional tissues. Unlike the HARV bioreactor, the HFB employs a variable hydrofocusing force that can control the movement, location and removal of suspended cells, three-dimensional tissues, and air bubbles from the bioreactor. Only gentle mixing is required to distribute nutrients and oxygen. These factors allow higher concentrations and densities to be achieved in a low G environment. Additionally, since the cells do not need to maintain the same surface forces that they require in Earth-normal gravity, they can divert more energy sources for growth and differentiation, the biosynthesis of more products, or even novel products. This allows the ability to impose variable gravity on these cell systems and the means to test the consequences of increasing or decreasing G on bioactive compound synthesis.

Research with mammalian cells in rotating low shear bioreactors is mainly focused on tissue engineering, three-dimensional in vitro tissue models for new drug development and testing; vaccine production; and for ensuring astronaut health (Anon., “Culturing a future,” Microgravity News 5(3)3-5 (1998); Unsworth, B. R., and Lelkes, P. I., Nature Medicine 4:901-906 (1998)). Previous conventional mammalian cell culture processes were incapable of simultaneously achieving sufficiently low shear stress, sufficient three-dimensional spatial freedom for cell growth and sufficiently long periods for critical cell interactions (with each other or substrates) to allow for adequate modeling of in vivo tissue structure. (See e.g., U.S. Pat. No. 5,308,764.) With the introduction of cylindrical horizontally rotating bioreactors, a stabilized environment was produced into which cells or tissues could be introduced, suspended, assembled, grown, and maintained with retention of delicate three-dimensional structural integrity. Bioreactors provide a means to culture red blood cells or skin in the event of astronaut trauma.

Unlike plant cells, mammalian cells do not have cell walls or large fluid-filled vacuoles. Both of these cell structures contribute to the shear-sensitive nature of plant cells. Therefore mammalian cells are not as sensitive to shear forces as plant cells. However, mammalian cell cultures are prone to pathogen contamination. As such, they require that antibiotics be added to culture media. Furthermore, plant cells naturally produce a host of medicinal compounds that can not be readily obtained from mammalian cell culture. Mammalian cells have to be genetically modified to produce bioactive compounds of interest. Therefore, plant cell culture offers a less expensive process by which a multitude of medicinal compounds can be produced.

Plant biomass production in an HFB can be rapid and can serve as a smaller, quicker way of growing plant cells for Advanced Life Support applications, where time, energy, and volume will be limiting factors. Long term space travel by humans may be limited by supplies of food, water, and oxygen. In one embodiment, HFBs can thus be used to culture plant cells to provide food and replenished air supplies for the spacecraft, or planetary colony. The success of a long term manned mission depends on efficient technologies enabling the needs of space crews to be met. Higher plant cells can provide food and oxygen, as well as recycled water in bioregenerative systems. Thus, the three-dimensional plant tissue model will support investigations into the role of gravity on three-dimensional, high-fidelity plant tissue growth and differentiation, and production of biomass and valuable medicinal bio-products.

Therefore, an aspect of the present invention is directed to a method for the continuous culture of plant cells in a hydrofocusing bioreactor. Unlike earlier shake-flask culturing methods, the bioreactor culturing system provides a low-shear environment for the culture of shear-force sensitive cells, such as plant cells. The hydrofocusing bioreactor distinguishes itself from other horizontally-rotating bioreactors in that it offers a hydrofocusing culture environment that allows for the co-location of particles within the culture chamber of the bioreactor and the efficient removal of metabolic wastes and air bubbles. The method promotes the growth of tissue-like, three-dimensional cell constructs. By the term “tissue-like, three-dimensional cell constructs” is meant cell tissue(s) that have three-dimensionality. The term “tissue-like, three-dimensional cell construct(s)” is used interchangeably with “tissue(s).”

Other cell types that have been commonly employed for the production of bioactive compounds of interest are fungal and bacterial cells. Fungal and bacterial cells also share the common characteristic of a cell wall with plant cells. Although fungal and bacterial cells are less sensitive to shear forces, they have not been previously grown in a hydrodynamic focusing environment. Like plant cells, fungal and bacterial cells form three-dimensional cell constructs on plated agarose media. On plated agarose media, fungal and bacterial three-dimensional cell constructs are commonly referred to as colonies. One result of culturing fungal and bacterial cells in an HFB bioreactor would be the production of three-dimensional fingal and bacterial constructs/colonies in suspension. These three-dimensional fungal and bacterial constructs/colonies could also be used for a variety of purposes including the production of bioactive compounds of interest on Earth and in space, a means to produce higher yields of bioactive compounds of interest and a means to investigate cell communication between fungal and bacterial cells within a three-dimensional fungal and bacterial construct/colony. In the HFB, fungal and bacterial cells experience all of the advantages that plant cells experience in a hydrodynamic focusing environment. Therefore, another aspect of the present invention is directed to a method for the continuous culture of fungal or bacterial cells in a hydrofocusing bioreactor.

Another aspect of the present invention is directed to a method for producing one or more bioactive compounds by continuously culturing plant, fungal or bacterial cells in a hydrofocusing bioreactor. Again, unlike earlier shake-flask culturing methods, the bioreactor culturing system provides a low-shear environment for the culture of plant cells, which are sensitive to shear forces. Furthermore, biomass and bioactive compound concentrations are increased by the culture of plant cells in bioreactors. Additionally, since the cells do not need to maintain the same surface forces that they require in Earth-normal gravity, they can divert more energy sources for growth and differentiation, the biosynthesis of more products, or even novel products.

By the term “biomass” is meant the grams of dry weight of cells per liter of culture. Dry weight of cells is determined by placing a sample containing cells from the bioreactor vessel onto pre-weighed filter paper, removing media by suction, washing the cells with water, drying the cells, and weighing them.

Some advantages of using plant cell suspension cultures for production of biologically active compounds are low raw material costs, capability of post-translational modification of proteins, and diminished risk of mammalian pathogen contamination. One embodiment is a method for increasing the production of one or more bioactive compounds in plant cells cultured in a hydrofocusing bioreactor, wherein the level of bioactive compounds produced is increased over the level of the same bioactive compounds produced via shake-flask culture. Another embodiment is a method for increasing the production of one or more bioactive compounds in plant cells cultured in a hydrofocusing bioreactor, wherein the level of bioactive compound produced is increased over the level of the same bioactive compound produced via shake-flask culture from about two-fold to about ten-fold. A preferred embodiment is a method for increasing the production of one or more bioactive compounds in plant cells cultured in a hydrofocusing bioreactor, wherein the level of bioactive compound produced is increased over the level of the same bioactive compound produced via shake-flask culture by ten-fold.

A preferred embodiment is a method for increasing the production of alkaloids in plant cells cultured in a hydrofocusing bioreactor compared to the levels of alkaloids produced in plant cells cultured in shake-flasks. Preferably, the alkaloids being produced are catharathine and serpentine. Large-scale cultivation of plant cells in bioreactors increases the biomass production much more rapidly than the whole plants that are grown in the field. Culture cycles of cell suspensions in bioreactors can be extended to weeks. Methods for increasing the biomass of plant cells cultured in a hydrofocusing bioreactor compared to the biomass of plant cells cultured in shake-flasks are also contemplated.

Similarly, low raw material costs are incurred for the production of bioactive compounds of interest using fungal or bacterial cell suspension cultures in the HFB. Therefore, another embodiment is a method for increasing the production of one or more bioactive compounds in fungal or bacterial cells cultured in a hydrofocusing bioreactor, wherein the level of bioactive compound produced is increased over the level of the same bioactive compound produced via shake-flask culture. A preferred embodiment is a method for increasing the production of one or more bioactive compounds in fungal or bacterial cells cultured in a hydrofocusing bioreactor, wherein the level of the bioactive compound produced is increased over the level of the same bioactive compound produced via shake-flask culture from about two-fold to about ten-fold.

Another embodiment is to a method for increasing the production of one or more bioactive compounds in induced plant cells cultured in a hydrofocusing bioreactor compared to levels of bioactive compounds in uninduced plant cells cultured in a hydrofocusing bioreactor. In one embodiment, the plant cells are induced with an abiotic agent added to the culture media, such as mannitol or polyvinylpyrrolidone. In another embodiment, the plant cells are induced with a biotic agent added to the media, such as an Aspergillum niger mycelium extract. The plant cells may also be induced with both abiotic and biotic inducing agents. Preferably, the plant cells are induced with both polyvinylpyrrolidone and Aspergillum niger mycelium extracts. By the term “biotic agent” is meant a compound produced by living organisms. By the term “abiotic agent” is meant a compound that has artificial origins. Abiotic agents that are known to stimulate bioactive compound production include NaCl, KCl, methyl jasmonate and jasmonic acid.

Like plant cells, the production of bioactive compounds in fungal and bacterial cells can be induced with the introduction of an abiotic or biotic agent to the culture medium. Therefore, another aspect of the present invention is directed to a method for increasing the production of one or more bioactive compounds in induced fungal or bacterial cells cultured in a hydrofocusing environment compared to levels of bioactive compounds in uninduced fungal or bacterial cells cultured in a hydrofocusing bioreactor. In one embodiment, the fungal cells are induced with an abiotic agent added to the culture media, such as methanol. Other abiotic agents that can be added to culture media to induce fungal cell secondary metabolite production are metals like cadmium, manganese, cobalt, boron and molybdenum. Butyrolactone I, a biotic agent, has been used to increase the production of desired secondary metabolites in filamentous fungus Aspergillus terreus. In another embodiment, the fungal cells are induced with a biotic agent. In a particular embodiment, the fungal cells are induced with a biotic agent added to the culture media, such as Butyrolactone I. In another embodiment, the fungal cells are induced with both biotic and abiotic agents. In one embodiment, the bacterial cells are induced with an abiotic agent added to the culture media, such as isopropyl-beta-D-thiogalactopyranoside (ITPG). Other abiotic agents that can be added to the culture media to induce bacterial cells are Mg⁺², Zn⁺, Mn⁺², Fe⁺ and DMSO. In another embodiment, the bacterial cells are induced with a biotic agent. In another embodiment, the bacterial cells are induced with both biotic and abiotic agents.

Another aspect of the present invention is directed to a method for assaying the presence of one or more bioactive compounds by continuously culturing plant, fungal or bacterial cells in a hydrofocusing bioreactor. The low-shear environment of the hydrofocusing bioreactor offers a better culture environment for plant cells compared to the shake-flask and impeller-driven bioreactor systems previously used. The hydrofocusing bioreactor allows for the co-location of particles within the culture chamber of the bioreactor and the efficient removal of metabolic wastes, air bubbles, media and cell culture samples. This is particularly advantageous in assaying bioactive compounds that are secreted into the media. The bioactive compounds secreted into the media can easily be removed through the sampling port and assayed for activity. The cell culture samples can also be removed through the sampling port and harvested for assays. Obtaining cell culture samples from the HFB is much easier than in earlier first-generation horizontally-rotating bioreactors.

Solvent extraction is a technique commonly used to recover a bioactive compound from either a solid or liquid. The sample is contacted with a solvent that will dissolve the solutes of interest. Some extraction techniques involve partition between two immiscible liquids; others involve either continuous extractions or batch extractions. Typical procedures for detecting and recovering bioactive compounds include filtering the culture and extracting the filtrate with the same volume of ethyl acetate. The organic phase is evaporated in vacuum. This extraction process can be repeated multiple times.

Dried and pulverized plant materials may be soaked in an organic solvent to extract the bioactive compounds. Bioactive compounds are generally recovered isocratically through the use of reverse phase high-performance liquid chromatography (HPLC) with UV detection at 280 nm. Optimum resolution of bioactive compounds occurs when an HPLC mobile phase consists of a methanol to 1% aqueous acetic acid ratio of 40:60 v/v, at pH 4.

Another aspect of the invention is directed to a process for producing tissue-like, three-dimensional plant, fungal or bacterial cell constructs in a hydrofocusing bioreactor. The hydrofocusing bioreactor allows for the formation of tissue-like, three-dimensional plant cell constructs, unlike shake-flask and impeller-driven culture methods previously used. Furthermore, the hydrofocusing bioreactor is better equipped to promote the survival of tissue-like, three-dimensional plant cell constructs compared to other horizontally-rotating bioreactors because the hydrofocusing bioreactor can efficiently remove metabolic wastes and air bubbles that are detrimental to the survival of plant cells.

Another aspect of the present invention is directed to a tissue-like, three-dimensional plant cell construct grown in a hydrofocusing bioreactor. Plant cells experience cytoskeleton reorganization and actin degradation when grown in microgravity. Plant cells grown in microgravity also exhibit swollen chloroplasts compared to plant cells grown under Earth-gravity conditions. Therefore, one embodiment of the invention is a tissue-like, three-dimensional plant cell construct that has a reorganized and degraded cytoskeleton and swollen chloroplasts.

By the term “continuous culture” is meant the growth of cells in culture medium in a culture chamber, whereby removal of all or some of the medium in a culture vessel occurs while the cells are retained in the culture chamber. Methods for growing plant tissue culture cells are known to those of skill in the art. Thus, for continuous culture, cells are cultured in medium that is exchanged after a period of time for fresh medium. Cells are not removed from the culture chamber during the medium exchange.

The methods described above contemplate tissue culture cells that are derived from many different plants. The methods thus have use over a broad range of types of plants, including but not limited to the species from the genera Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa (e.g., solanaceae, belladonna), Capsicum, Datura (e.g., solanaceae, metel), Hyoscyamus (e.g., niger, albus), Lycopersicon, Nicotiana, Solanum (e.g., Xanthocarpum), Petunia, Digitalis (e.g., lanata), Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Zea, Avena, Hordeum, Secale, Triticum, Catharanthus (e.g., roseus G. Don), Scopolia (e.g., solanaceae), Duboisia (e.g., solanaceae), Taxus (e.g., baccata), Nothapodytes (e.g., foetida), Artemisia (e.g., annua), Santalum (e.g., album L.), Lithospermum (e.g., erythrorhizon), Sorghum, Aloe (e.g., barbadensis), Cinchona (e.g., ledgeriana), Dioscorea (e.g., deltoida, composita), Glycyrrhiza (e.g., glabra), Panax (e.g., ginseng), Papaver (e.g., somniferum), Rheum (e.g., officinale), Rouwolfia (e.g., serpentina), Eucalyptus (e.g., globulus), Eugenia (e.g., caryophyllata), Jasminum, Lavandula (e.g., angustffolia), Mentha (e.g., pzerita), Pelargonium, Thaumatocoeus (e.g., danielli), and Vetiver.

In a preferred embodiment, the plant cells cultured are Catharanthus roseus G. Don plant cells. The plant cells cultured may be derived from cotyledons, hypoctyls, epicotyls, shoot tips, root tips, stem and leaf calli, as well as root and hairy-root plant cell cultures. The plant cells cultured may also be transgenic plant cells. By the term “transgenic plant cell” is meant a plant cell whose genome has been altered by the transfer of a gene or genes from another species or breed. During culture in the HFB, the plant cells co-locate to produce three-dimensional plant cell tissue-like constructs. The three-dimensional plant cell tissues have a length of about 4 mm to about 10 mm. Preferably, the three-dimensional plant cell tissues have a length of about 4, 5, 6, 7, 8, 9 or 10 mm.

By the term “plant cells” is meant cells derived from any part of a plant, including shoot vegetative organs/structures (e.g., leaves, stems and tubers), roots, flowers and floral organs structures (e.g., bracts, sepals, petals, stamens, carpels, anthers and ovules), seed (including embryo, endosperm and seed coat) and fruit (the mature ovary), or plant tissue (e.g., vascular tissue, ground tissue, and the like) or particular cells (e.g., guard cells, egg cells, trichomes, and the like), and progeny of the same. The class of plant cells that can be used in the method of the invention is generally as broad as the class of higher and lower plants amenable to cell culturing techniques, including angiosperms (monocotyledonous and dicotyledonous plants), gymnosperms, ferns, and multicellular algae. It includes plant cells of a variety of ploidy levels, including aneuploid, polyploid, diploid, haploid and hemizygous plants. Plant cells can also be subsequently propagated as callus, plant cells in suspension, organized tissue and organs.

Tissue cultures derived from the plant tissue of interest can be established using well-known methods for establishing and maintaining plant tissue cultures. (e.g., Trigiano R. N. and Gray D. J. (1999), “Plant Tissue Culture Concepts and Laboratory Exercises”, ISBN: 0-8493-2029-1; Herman E. B. (2000), “Regeneration and Micropropagation: Techniques, Systems and Media 1997-1999”, Agricell Report). Typically, the plant material is surface-sterilized prior to introducing it to the culture medium. Any conventional sterilization technique, such as chlorinated bleach treatment can be used. Under appropriate conditions plant tissue cells form callus tissue, which may be grown either as solid tissue on solidified medium or as a cell suspension in a liquid medium.

The methods described above also contemplate tissue culture cells that are derived from many different bacteria. The methods thus have use over a broad range of types of bacteria, including but not limited to the species from the genera Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia (e.g., coli), Salmonella, Bacillus, Streptomyces, Shewanella, Lactococcus, Streptococcus, Oenococcus, Lactosphaera, Trichococcus, Pediococcus, Rhodococcus, Alcaligenes, Arthrobacter, Bacteridium, Brevibacterium, Corynebacterium, Agrobacterium, Micrococcus, Comamonas, Erwinia, Xanthomonas, Azoarcus and Pseudomonas. The methods also contemplate the use of transgenic fungal cells.

The methods described above also contemplate tissue culture cells that are derived from many different fungi. The methods thus have use over a broad range of types of fungi, including but not limited to the species from the genera Agaricus, Agrocybe, Armillaria, Clitocybe, Collybia, Conocybe, Coprinus, Flammulina, Giganopanus, Gymnopilus, Hypholoma, Inocybe, Hypsizygus, Lentinula, Lentinus, Lenzites, Lepiota, Lepista, Lyophyllum, Macrocybe, Marasmius, Mycena, Omphalotus, Panaeolus, Panellus, Pholiota, Pleurotus, Pluteus, Psathyrella, Psilocybe, Schizophyllum, Sparassis, Stropharia, Termitomyces, Tricholoma, Volvariella, Polyporaceae, Albatrellus, Antrodia, Bjerkandera, Bondarzewia, Bridgeoporus, Ceriporia, Coltricia, Daedalea, Dentocorticium, Echinodontium, Fistulina, Flavodon, Fomes, Fomitopsis, Ganoderma, Gloeophyllum, Grifola, Hericium, Heterobasidion, Inonotus, Irpex, Laetiporus, Meripilus, Oligoporus, Oxyporus, Phaeolus, Phellinus, Piptoporus, Polyporus, Schizopora, Trametes, Wolfiporia, Auricularia, Calvatia, Ceriporiopsis, Coniophora, Cyathus, Lycoperdon, Merulius, Phlebia, Serpula, Sparassis Stereum, Cordyceps, Morchella, Tuber, Peziza, Tremella, Acaulospora, Alpova, Amanita, Astraeus, Athelia, Boletinellus, Boletus, Cantharellus, Cenococcum, Dentinum, Gigaspora, Glomus, Gomphidius, Hebeloma, Lactarius, Paxillus, Piloderma, Pisolithus, Rhizophagus, Rhizopogon, Rozites, Russula, Sclerocytis, Scleroderma, Scutellospora, Suillus, Tuber, Phanerochaete (e.g., chrysosporium, sordida), Actinomyces, Alternaria, Aspergillus, Botrytis, Candida, Chaetomium, Chrysosporium, Cladosporium, Cryptococccus, Dactylium, Doratomyces (e.g., stysanus), Epicoccum, Fusarium, Geotrichum, Gliocladium, Humicola, Monilia, Mucor, Mycelia Sterilia, Mycogone, Neurospora, Papulospora, Penicillium, Rhizopus, Scopulariopsis, Sepedonium, Streptomyces, Talaromyces, Torula, Trichoderma, Trichothecium, Verticillium. Metarhizium, Beauveria, Paecilomyces, Verticillium, Hirsutella, Aspergillus, Akanthomyces, Desmidiospora, Hymenostilbe, Mariannaea, Nomuraea, Paraisaria, Tolypocladium, Spicaria, Botrytis, Rhizopus, Entomophthoracae, Phycomycetes, Saccharomyces and Cordyceps. The methods also contemplate the use of transgenic bacterial cells.

The methods described above also contemplate tissue culture cells that are encapsulated in microspheres or microcapsules. Alginate microspheres are one example of polysaccharide-based microspheres. Microspheres serve to encapsulate cells and can produce high density cultures protected from shear damage in flow or stirred systems. For example, scopolin-producing free Nicotiana tabacum cell suspensions accumulate scopolin within cytoplasmic compartments. Cell disruption is necessary to recover the scopolin molecules. Nicotiana tabacum cells that are immobilized within calcium-alginate microspheres excrete significant amounts of scopolin. The scopolin molecules diffuse throughout the gel matrix and into the culture media. In this manner, a large fraction of scopolin can be recovered from the culture media without cell disruption. Further, immobilized Nicotiana tabacum cells produce more scopolin (3.8 mg/g fresh weight biomass [from culture media]) than free cell suspensions (0.2 mg/g fresh weight biomass [intracellular]) (Gilleta, F. et al., Enzyme Microb. Technol. 26:229-234 (2000)).

### Hydrodynamic Focusing Bioreactor (HFB)

The hydrofocusing bioreactor is a cell culture apparatus that employs hydrodynamic focusing to simulate microgravity. The HFB contains a rotating, cell culture chamber and an internal viscous spinner. The chamber and spinner can rotate at different speeds in either the same or opposite directions. Rotation of the chamber and viscous interaction at the spinner generate a hydrofocusing force. Adjusting the differential rotation rate between the chamber and spinner controls the magnitude of the hydrofocusing force and the co-location of contents within the culture chamber

By the term “bioreactor” is meant an apparatus, such as a large fermentation chamber, for growing organisms such as bacteria, yeast, plant or mammalian cells that are used in the biotechnological production of substances such as pharmaceuticals, antibodies, or vaccines, or for the bioconversion of organic waste.

By the term “hydrofocusing bioreactor” is meant a bioreactor that relies on the principle of hydrodynamic focusing to control the movement of contents within the culture chamber of the bioreactor. By the term “hydrodynamic focusing” is meant relating to, or operated by the force of liquid in motion to control the movement of contents within the culture chamber of the bioreactor. The HFB offers a unique hydrofocusing capability that enables the creation of a low-shear culture environment simultaneously with the “herding” of suspended cells, tissue assemblies, and air bubbles.

By the term “culture chamber” is meant the enclosed space or compartment in which plant cells are cultured. In one embodiment of the present invention, the hydrofocusing bioreactor is a horizontally-rotating bioreactor. In another embodiment, the bioreactor has both a culture chamber and an internal viscous spinner. The culture chamber and the internal viscous spinner can be horizontally rotated to produce a hydrofocusing force on the contents of the culture chamber or the culture chamber can be rotated in the same direction as the internal viscous spinner. The culture chamber can be horizontally-rotated at a rate from about 1 RPM to about 30 RPM in 1 RPM increments. The internal viscous spinner can be horizontally-rotated from about 1 RPM to about 99 RPM, in 1 RPM increments.

By the term “differential rate” is meant the difference between the rotational rate of the culture chamber and the rotational rate of the inner viscous spinner. The bioreactor can have a differential rate from about 1 RPM to about 129 RPM. Preferably, the bioreactor differential rate is 25 RPM. For plant cell aggregates larger than 8 mm, a higher differential rate of 40 RPM is required to keep tissue assemblies from breaking apart. A differential rate from about 15 RPM to about 25 RMP is preferred for culturing fungal and bacterial cells in the HFB. The culture chamber can also be rotated in the opposite direction as the internal viscous spinner. The rate of rotation for the culture chamber may be higher than the rate of rotation of the internal viscous spinner, lower than the rate of rotation of the internal viscous spinner or the same as the rate of rotation of the internal viscous spinner. The bioreactor may also have a dome-shaped culture chamber.

The hydrofocusing bioreactor culture chamber has a volume between about 10 mL and about 10 L. (See e.g., PCT publication WO 00/00586.) Small and medium scale laboratory cultures can be performed in culture chambers of 100 mL, 250 mL, and 500 mL volumes. In one embodiment, the bioreactor has a culture chamber volume of about 160 mL. In another particular embodiment, the bioreactor has a culture chamber volume of about 40 mL. Larger preparative scale cultures can be performed in culture chambers of 1 L, 5 L, and 10 L volumes. In another embodiment, the bioreactor has a culture chamber volume of 1, 2, 3, 4, 5, 6, 7, 8, 9 or 10 L. The bioreactor culture chamber can have perfusion ports to allow for gas exchange. The bioreactor culture chamber can have a sample port that allows for extraction of media, cells or air.

In one embodiment, the bioreactor allows co-location of cells with similar or differing sedimentation properties in a similar spatial region within the culture chamber. In another embodiment, the bioreactor allows freedom for the three-dimensional spatial orientation of plant, fungal or bacterial cell tissues formed by the culturing of the plant, fungal or bacterial cells. In yet another embodiment, low shear and essentially no relative motion of said culturing environment is observed with respect to the walls of the culture chamber. The resulting force, within the bioreactor suspends cells in a low-shear environment such that a maximum force of 0.01 dyne/cm² is experienced by the plant cell walls. Another aspect of the invention is to a method for culturing plant cells in a HFB, whereby the resulting force within the bioreactor suspends cells in a low-shear environment such that a maximum force of 0.5 dynes/cm² is experienced by the plant cell walls.

### Growth Conditions

Plant cells can be grown in the bioreactor under cool-white fluorescent light. In certain embodiments, the plant cells are grown under cool-white fluorescent light with a light output from about 4 to about 12 W/m². In particular embodiments, the plant cells are grown under cool-white fluorescent light with a light output of 4 W/m². Plant cells can also be grown under cool-white fluorescent light with a light output of 12 W/m². Preferably, plant cells are grown in the absence of light, i.e. in the dark.

Plant cells can be continuously cultured for a period of at least 20 days in the absence of antibiotics. In the presence of antibiotics, plant cells can be continuously cultured for a period of at least 35 days. In one embodiment of the present invention, the plant cells are continuously cultured from about 3 to about 35 days. In another embodiment, the plant cells are continuously cultured for 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days. In a preferred embodiment, the plant cells are continuously cultured for at least 3 days. In a particular embodiment, the plant cells are continuously cultured for at least 5 days. In a particular embodiment, the plant cells are continuously cultured for at least 7 days. In a particular embodiment, the plant cells are continuously cultured for at least 14 days. In a particular embodiment, the plant cells are continuously cultured for at least 20 days. In a particular embodiment, the plant cells are continuously cultured for at least 28 days. In a particular embodiment, the plant cells are continuously cultured for at least 35 days.

Bacterial cells can be continuously cultured for a period of at least 1 day. In one embodiment of the present invention, the bacterial cells are continuously cultured from about 8 hours to about 7 days. In another embodiment, the bacterial cells are continuously cultured for at least 8 hours. In a particular embodiment, the bacterial cells are continuously cultured for at least 20 hours. In a particular embodiment, the bacterial cells are continuously cultured for at least 24 hours. In a particular embodiment, the bacterial cells are continuously cultured for at least 7 days.

Fungal cells can be continuously cultured for a period of at least 1 day. In one embodiment of the present invention, the fungal cells are continuously cultured from about 8 hours to about 7 days. In another embodiment, the fungal cells are continuously cultured for at least 8 hours. In a particular embodiment, the fungal cells are continuously cultured for at least 20 hours. In a particular embodiment, the fungal cells are continuously cultured for at least 24 hours. In a particular embodiment, the fungal cells are continuously cultured for at least 7 days.

Since the lag phase is shortened in fungal and bacterial cells cultured in the HFB and cell density increases rapidly in less than 7 days, antibiotics may not need to be added to the culture media. Therefore, in one embodiment, the culture media does not contain any antibiotics.

Oxygen and carbon dioxide are vital gases that are required by plant cells for respiration and photosynthesis. Similarly, oxygen is also important for the respiration of fingal and bacterial cells. It is contemplated that the media within the culture chamber of the bioreactor can be oxygenated. It is also contemplated that the metabolic waste products formed within the culture chamber of the bioreactor can be removed. Metabolic waste products can be removed through the sampling port of the HFB. By the term “metabolic waste products” is meant substances left over from metabolic processes, which cannot be used by the organism (they are surplus or have a lethal effect), and must therefore be excreted. Metabolic waste products include CO₂, O₂, phosphates, sulfates and indoles. In one embodiment, air bubbles formed within the culture chamber of the bioreactor can be removed. Air bubbles can be removed through the sampling port of the HFB.

Nutrient-depleted media can also be removed. By the term “nutrient-depleted media” is meant media that is depleted of essential carbohydrates, amino acids, fatty acids, vitamins and minerals required for cell growth. In a particular embodiment, the method of culturing the plant, fingal or bacterial cells includes filling the culture chamber with an oxygen rich nutrient media and plant, fungal or bacterial cells of one or more distinct types to establish a culturing environment within the culture chamber. In another embodiment, nutrient-depleted media can be replaced with oxygen rich nutrient media. In another embodiment, nutrient-depleted media can be replaced with oxygen rich nutrient after 4 days of culture.

A number of suitable culture media for plant callus induction and subsequent growth on aqueous or solidified media are known. The term “culture media” is used interchangeably with “nutrient media” and refers to a liquid or gelatinous substance containing nutrients in which microorganisms or tissues are cultivated for scientific purposes. Exemplary plant media include standard growth media, many of which are commercially available (e.g., Sigma Chemical Co., St. Louis, Mo.). Examples include Schenk-Hildebrandt (SH) medium, Linsmaier-Skoog (LS) medium, Murashige and Skoog (MS) medium, Gamborg's B5 medium, Nitsch & Nitsch medium, White's medium, and other variations and supplements well known to those of skill in the art (See, e.g., Plant Cell Culture, Dixon, ed IRL Press, Ltd. Oxford (1985) and George et al., Plant Culture Media, Vol 1, Formulations and Uses Exegetics Ltd. Wilts, UK (1987)). Exemplary fungal media include Yeast Peptone Dextrose (YPD) medium, Sabouraud Dextrose (SD) medium, Sabouraud Maltose (SM) medium, and other variations and supplements well known to those of skill in the art. (See, e.g., DIFCO Manaul, ed Difco Laboratories, Michigan (1977)). Exemplary bacterial media include Luria Broth (LB) medium, Dubos Broth (DB) medium, Terrific Broth (TB) medium, and other variations and supplements well known to those of skill in the art. (See, e.g., DIFCO Manaul, ed Difco Laboratories, Michigan (1977)). By the term “growth medium” is meant culture medium which allows growth and division of plant, fungal or bacterial cells. Growth medium, generally speaking, is not optimal for production of protein from an inducible promoter.

Further, attachment materials can supplement the culture media. By “attachment materials” is meant materials that provide a surface onto which cells in cell culture suspensions can attach. An example of attachment materials includes microcarrier beads. These beads provide a support for the growth, maintenance and differentiation of various tissue and cell types. Microcarrier beads are especially useful for the growth of anchorage-dependent species of plant cells. Gelatin-coated microcarrier beads provide an optimal substrate for anchorage-dependent plant cells resulting in rapid and strong attachment. These beads are coated with gelatin from porcine skin and are available in two densities: 1.02 and 1.03 g/cm³. Examples of useful microcarrier beads are those having product numbers M8778, M8903 and M9560, which can be obtained commercially (Sigma Aldrich).

Using the HFB bioreactor, the amount of antibiotics needed to limit undesired bacterial growth in plant cell cultures is reduced or eliminated as compared to culturing plant cells in shake-flasks. Samples can be taken from the bioreactor vessel during the culture of plant cells, as long as precautions are taken to maintain the sterility of the culture. Those of skill in the art are familiar with techniques to maintain sterility. Aseptic techniques include taking samples directly from the bioreactor vessel in a laminar flow hood. Thus, the hydrofocusing bioreactor may be operated within a laminar flow hood. In one embodiment of the present invention, the concentration of antibiotics added to culture media to limit bacterial growth is limited to about 0.1 mg/L to about 1 mg/L of culture. In another embodiment, the concentration of antibiotics added to culture media to limit bacterial growth is limited to about 0.5 mg/L.

To achieve optimal production of bioactive compounds, one must monitor the culture medium within the bioreactor culture chamber and determine whether the appropriate conditions for cell growth are present. These growth conditions include the presence of nutrients such as carbohydrates, amino acids, fatty acids, vitamins and minerals, oxygen, carbon dioxide, and the appropriate temperature and pH for cell growth. In one embodiment, the method directed to producing bioactive compounds further includes monitoring the fluid culture medium within the culture chamber. With the HFB, the pH, temperature and the dissolved oxygen levels of the culture medium can also be monitored. The method can include monitoring the pH, temperature and the dissolved oxygen levels of the culture medium online within the bioreactor culture chamber or after assaying samples of the medium withdrawn from the culture chamber.

Media is exchanged from the bioreactor vessel for a variety of reasons, including to induce protein, carbohydrate, lipid, nucleic acid, metabolite or chemical production, to harvest the bioactive compound of interest, or to restart growth of the cells after nutrient depletion. One of skill in the art will understand that media exchange can be carried out in a variety of ways. Sterile media can be added after filtration through a sterile filter. Fresh medium can be added to the cells. The fresh medium may have the same components or different components than the original unspent medium. For example, “induction medium” may be exchanged with “growth medium,” or the reverse may also occur. By the term “induction medium” is meant culture medium which provides a culture environment that activates transcription or alleviates repression of transcription from an inducible promoter. By the term “inducing agent” is meant to describe biotic or abiotic compounds that allow for the enhanced production of a bioactive compound of interest.

When cells produce heterologous protein, the pH of the medium rises as expressed protein levels increase. The methods further include adjusting the pH of the fluid culture medium. pH measurement is thus conveniently used as an indicator of protein production and as an indicator of when the heterologous protein can be harvested or when media can most optimally be exchanged back to growth medium if an induction medium is used. In one embodiment, the upper pH limit for medium exchange will be less than pH 8.5. In another embodiment, the upper pH limit for medium exchange will be less than pH 8.0. As most plant tissue cultures are grown between about pH 5.2 and about pH 5.8, other embodiments include medium exchange for media that has a pH of about 5.2, 5.3, 5.4, 5.5, 5.6, 5.7 or 5.8. As most fungal cultures are grown between about pH 5 and about pH 8, other embodiments include medium exchange for media that has a pH of about 5, 6, 7 or 8. As most bacterial cultures are grown between about pH 7 and about pH 8, other embodiments include medium exchange for media that has a pH of about 7.5. One of skill in the art will recognize that the pH value for optimal protein, metabolite or chemical production will vary with the culture conditions, the type of cells, and the bioactive compound being produced. Measurement of pH is well known to those of skill in the art. The pH can be measured using a pH electrode in combination with a device for reporting the pH. The pH can also be detected using pH sensitive dyes, usually bound to a paper support. The pH electrodes, pH meters, and pH paper are all commercially available from, for example, Fisher Scientific, Inc., and Broadley-James Corporation. The bioreactor will preferably include means to measure pH levels in the culture media.

Optimal plant, fungal or bacterial cell growth and production of bioactive compounds requires that the culture media temperature be regulated. In one embodiment, the method includes adjusting the temperature of the fluid culture medium. In another embodiment, the method includes adjusting the temperature of the culture medium to a fixed temperature from about 25° C. to about 35° C. In a preferred embodiment, the method further includes adjusting the temperature of the culture medium to 25° C. In another preferred embodiment, the method includes adjusting the temperature of the culture medium to 35° C. In another embodiment, the method includes adjusting the temperature of the culture medium to a fixed temperature from about 15° C. to about 37° C. In a preferred embodiment, the method includes adjusting the temperature of the culture medium to 15° C. In a preferred embodiment, the method includes adjusting the temperature of the culture medium to 30° C. In another preferred embodiment, the method includes adjusting the temperature of the culture medium to 37° C. Those of skill in the art will appreciate that optimal growth conditions will be different for tissue culture cells derived from different plant, fungal and bacterial species and will know to adjust culture conditions accordingly.

Cells are grown under sterile conditions with controlled dissolved O₂ levels. One of skill in the art would know how to measure dissolved oxygen levels in media, and how to use those levels to determine the rate of oxygen consumption over time. Dissolved oxygen sensors are commercially available from, for example, Broadley-James Corporation and Mettler Toledo Corporation. In one embodiment, the method includes adjusting the dissolved oxygen levels of the culture medium. In a particular embodiment, the dissolved oxygen levels in the culture medium can be elevated by using a bubble-trap oxygenator. The bioreactor will preferably include means to measure dissolved O₂ levels. Measurements can be taken online, within the bioreactor culture chamber or measurements can be taken offline, after samples of the medium have been withdrawn from the culture chamber, however, online measurements are preferred. These operations are included with HFB bioreactors commercially available from, for example, Celdyne Corp.

The requirements for O₂ may vary from one plant, fungal or bacterial species to another. Oxygen must be supplied continuously to provide adequate aeration since it affects metabolic activity, energy supply and anaerobic conditions. The available oxygen for plant cells in culture is determined by the oxygen transfer coefficient (kLa) and includes the proportion of O₂ that dissolves in water. Dissolved O₂ depletion that occurs as a result of the growing biomass' metabolic activity can affect the culture yield. Plant cells have a lower metabolic rate than microbial cells and a slower doubling time. Therefore, they require a lower dissolved 02 supply. In general, high aeration rates appear to reduce biomass growth. The level of O₂ in conventional bioreactor cultures can be regulated by agitation or stirring and through aeration, gas flow, and air bubble size. The gas porous membrane in the HFB facilitates high kLa values leading to high cell growth rates. For plant cell cultures where dissolved O₂ is low (5-10%), the dissolved O₂ inhibits biomass growth and somatic embryogenesis, while high dissolved O₂ (60%) favors undifferentiated biomass growth. The percent oxygen concentration in the bioreactor was calculated from the measured dissolved oxygen level and was based on oxygen solubility in the growth medium at 28° C.

### Bioactive Compounds

Many types of bioactive compounds can be produced using the present invention. By the term “bioactive compound” is meant a substance that has an effect on living tissue. The bioactive compounds being produced include: proteins, carbohydrates, lipids, nucleic acids, metabolites, and chemicals. Some of the proteins of interest include without limitation, therapeutic proteins, antibodies, enzymes, protease inhibitors, transport proteins, storage proteins, protein toxins, hormones, and structural proteins. Since cells are retained in the chamber culture during continuous culture, the bioactive compound is preferably secreted into the medium. Bioactive compounds may be native to the plant, fungal or bacterial cell or encoded by genes endogenous to the plant, fungal or bacterial cell. Alternatively, bioactive compounds may be expressed from transgenic plant, fungal or bacterial cells. Transgenic plant, fungal or bacterial cells may carry a heterologous gene that encodes a protein of interest. Proteins expressed from heterologous genes may be engineered to include a signal peptide for secretion, if the protein is not normally secreted. In a preferred embodiment, the bioactive compound being produced is a chemical. In a particular embodiment, the bioactive compound being produced is a phyto-chemical. In one specific embodiment, the bioactive compound being produced is an aromatic compound. In another specific embodiment, the chemical being produced is an alkaloid.

Generally, two basic types of metabolites are synthesized in cells, i.e., those referred to as primary metabolites and those referred to as secondary metabolites. A primary metabolite is any intermediate in, or product of the primary metabolism in cells. The primary metabolism in cells is the sum of metabolic activities that are common to most, if not all, living cells and are necessary for basal growth and maintenance of the cells. Primary metabolism thus includes pathways for generally modifying and synthesizing certain carbohydrates, proteins, fats and nucleic acids, with the compounds involved in the pathways being designated primary metabolites.

In contrast, hereto, secondary metabolites usually do not appear to participate directly in growth and development. They are a group of chemically very diverse products that often have a restricted taxonomic distribution. Secondary metabolites normally exist as members of closely related chemical families, usually of a molecular weight of less than 1500 Dalton, although some bacterial toxins are considerably longer. Secondary plant metabolites include, e.g., alkaloid compounds (e.g., terpenoid indole alkaloids, tropane alkaloids, steroid alkaloids, polyhydroxy alkaloids), phenolic compounds (e.g., quinines, lignans and flavonoids), terpenoid compounds (e.g., monoterpenoids, iridoids, sesquiterpenoids, diterpenoids and triterpenoids). In addition, secondary metabolites include small molecules (i.e., having a molecular weight of less than 600), such as substituted heterocyclic compounds which may be monocyclic or polycyclic, fused or bridged.

Many plant secondary metabolites have value as pharmaceuticals. Plant pharmaceuticals include, e.g., taxol, digoxin, colchicines, codeine, morphine, quinine, shikonin, ajmalicine and vinblastine. The definition of “alkaloids,” of which more than 12,000 structures have been described already, includes all nitrogen-containing natural products which are not otherwise classified as peptides, non-protein amino acids, amines, cyanogenic glycosides, glucosinolates, cofactors, phytohormones or primary metabolites (such as purine and pyrimidine bases). “Flavonoids” are defined as a class of secondary metabolites derived from a phenylbenzopyrone chemical structure.

A variety of clinically beneficial secondary metabolites are also produced by fungi. Beta-lactam antibiotics penicillin and cephalosporin, the antifungal antibiotic griseofulvin and the pharmacologically active compounds known as the ergot alkaloids are all examples of secondary metabolites that can be produced by fungi.

The presence of bioactive compounds made by plant, fungal or bacterial cells can be assayed. Bioactive compounds that are secreted into the media can be collected with media through the sampling port. Bioactive compounds that are retained in plant, fungal or bacterial cells can be collected in cell culture samples through the sampling port. In one embodiment of the present invention, the bioactive compounds being assayed are selected from the group consisting of: proteins, carbohydrates, lipids, nucleic acids, metabolites, and chemicals. In one embodiment, the bioactive compound being assayed is a chemical. In another embodiment, the chemical being assayed is an alkaloid. Bioactive compounds in plant, fungal or bacterial cells obtained with media, as well as bioactive compounds obtained by harvesting cell culture samples, can be purified and concentrated by methods known to those of skill in the art. In one embodiment, the presence of bioactive compounds can be determined by purifying the bioactive compounds from cell lysates or other complex mixtures through reverse phase HPLC, capillary electrophoresis, ion exchange, or size exclusion chromatography.

In another embodiment, the bioactive compound is a protein. In one embodiment, the protein can be assayed by its level of expression. In another embodiment, the protein can be assayed by determining its catalytic activity. In another embodiment, the protein can be assayed by determining its ability to bind to other proteins and small molecules by measuring its dissociation constant (K_{d}). By the term “dissociation constant” is meant the equilibrium constant for a reversible dissociation reaction. By the term “equilibrium constant” is meant the ratio of concentrations of reactants and products when equilibrium is reached in a reversible reaction. By the term “equilibrium” is meant the state at which rate of the forward chemical reaction equals the rate of the reverse chemical reaction.

When the bioactive compound is a protein from a transgenic plant cell, DNA constructs may be introduced into the genome of the desired plant host by a variety of conventional techniques. For example, the DNA constructs may be introduced directly into the genomic DNA of the plant cell using techniques such as electroporation and microinjection of plant cell protoplasts, or the DNA constructs can be introduced directly to plant tissue using ballistic methods, such as DNA particle bombardment.

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. (See e.g., Horsch et al. Science 233:496 498 (1984); Fraley et al. Proc. Natl. Acad. Sci. USA 80:4803 (1983).)

When the bioactive compound is a protein from a transgenic fungal or bacterial cell, DNA constructs may be introduced either in the form of a plasmid vector into the desired fungal or bacterial cell host or into the genome of the desired fungal or bacterial cell host by a variety of conventional techniques. For example, DNA constructs may be introduced directly into the fungal or bacterial cells as plasmids using techniques such as electroporation and heat-shock transformation.

Methods are available to ensure that the bioactive compounds of interest are being made correctly by the plant, fungal or bacterial tissue culture cells. Immunological detection can conveniently be used to detect the protein of interest. In addition, depending on the nature of the bioactive compound, functional assays can be designed to detect the presence of a bioactive compound. If appropriate, assays may be performed to determine whether proteins of interest are post-translationally modified.

If an appropriate antibody is available, immunoassays can be used to qualitatively or quantitatively analyze the bioactive compounds produced by the present invention. A general overview of the applicable technology can be found in Harlow & Lane, Antibodies: A Laboratory Manual (1988).

The proteins of interest can be detected and/or quantified using any of a number of well recognized immunological binding assays (See, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7.sup.th ed. 1991). Immunological binding assays (or immunoassays) typically use an antibody that specifically binds to an antigen of choice. The antibody may be produced by any of a number of means well known to those of skill in the art and as described in Harlow & Lane, Antibodies: A Laboratory Manual (1988).

Western blot (immunoblot) analysis may be used to detect and quantify the presence of a protein of interest in the sample. Western blot analysis can further be used to ensure a full length protein has been produced. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the protein of interest. The antibodies may be directly labeled or alternatively may be subsequently detected using labeled secondary antibodies (e.g., labeled sheep anti-mouse antibodies) that specifically bind to the primary antibodies.

Secondary metabolites can be assayed, intracellularly or in the extracellular space, by methods known in the art. Such methods comprise analysis by thin-layer chromatography, high pressure liquid chromatography, capillary chromatography, (gas chromatographic) mass spectrometric detection, radioimmunoassay (RIA) and enzyme immuno-assay (ELISA).

Many different bioactive compounds can be expressed using the present invention; thus, many different assays for functional compounds may be employed. One of skill in the art will be aware of the particular assay most appropriate to determine the functional activity of the expressed bioactive compound.

By the term “increased production” is meant that the level of one or more bioactive compounds of interest may be enhanced by at least 20%, 30%, 40%, 50%, 60%, 70%, 80%, 90% or at least 100% relative to the non-induced plant, fungal or bacterail cell culture or the plant, ftngal or bacterial cell culture grown in shake-flasks. An increased production of a bioactive compound can result in a detection of a higher level of the compounds in the medium of the plant, fungal or bacterial cell culture. Alternatively, a higher level of bioactive compounds can be detected inside the plant, fungal or bacterial cells. For example, a higher level of bioactive compounds can be detected inside the plant cell vacuole.

All references cited in the Examples are incorporated herein by reference in their entireties.

### EXAMPLES

### Example 1

### Operation of the Hydrofocusing Bioreactor (HFB)

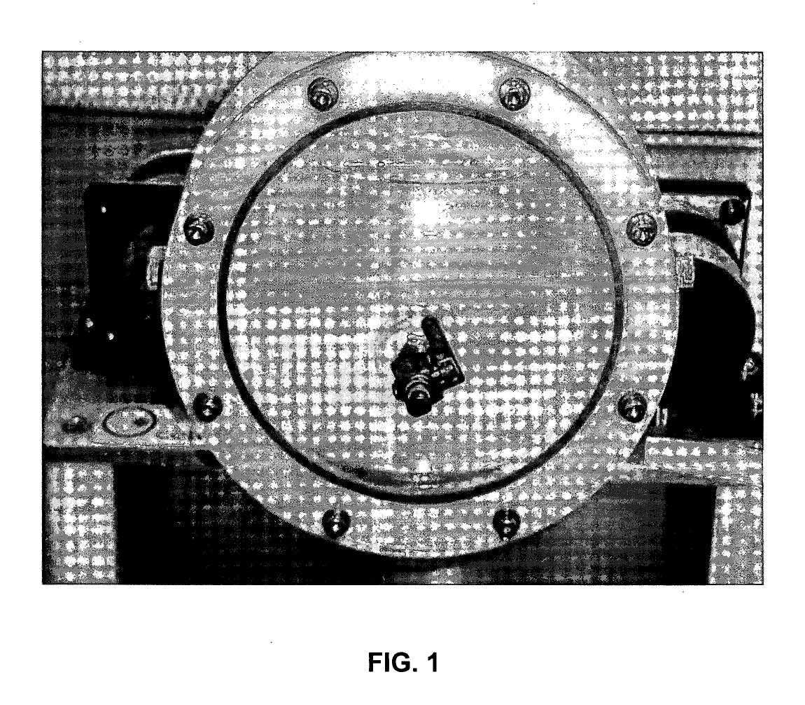

The HFB is an enabling technology for three-dimensional cell culture and tissue engineering investigations both in laboratories on Earth and on orbiting spacecraft. The HFB used in establishing Periwinkle cell suspension cultures contains a rotating, dome-shaped cell culture chamber with a centrally located sampling port and an internal viscous spinner (see FIG. 1). The chamber and spinner can rotate at different speeds in either the same or opposite directions. Rotation of the chamber and viscous interaction at the spinner generate a hydrofocusing force. Adjusting the differential rotation rate between the chamber and spinner controls the magnitude of the force. The HFB is equipped with a membrane for diffusion gas exchange to optimize gas/oxygen supply. Under the microgravity conditions of the HFB, at any given time, gravitational vectors are randomized and the shear stress exerted by the fluid on the synchronously moving particles is minimized. These simulated microgravity conditions facilitate spatial co-location and three-dimensional assembly of individual cells into large tissues (See e.g., Wolf, D. A. and Schwartz, R. P., Analysis of gravity-induced particle motion and fluid perfusion flow in the NASA-designed rotating zero-head-space tissue culture vessel., Washington D.C., NASA Tech Paper 3134, (1991).) In promoting three-dimensional tissue culture, an average shear value of 0.001 dynes/cm² was estimated for a rotation rate of 10 RPM. (See, e.g., Gonda, S. R. and Spaulding, G. F., Hydrofocusing Bioreactor for Three-Dimensional Cell Culture, NASA Tech Brief MSC-22538, Washington D.C. (2003).)

The HFB model used to establish Periwinkle cell suspension cultures is the HFB-EM2, Celdyne, Inc., Houston, Tex., http://www.celdyne.com/home/index.html. This model is supplied with a 160 mL culture chamber and a differential spinner drive to facilitate the positional control of cells and tissues within the chamber. The chamber rotation rate can be set with crystal controlled accuracy from 1 to 30 RPM in 1 RPM increments. The spinner rotation rate is similarly adjustable from 1 to 99 RPM. The HFB is operated inside of a Laminar Flow Hood. Aseptic techniques are employed when adding culture medium or inoculum to the culture chamber. After culture medium or inoculum addition, air bubbles are extracted via the sampling port to ensure that the HFB culture chamber is air-tight.

### Example 2

### Establishing Periwinkle Cell HFB Cultures

In order to establish a continuous Periwinkle cell culture within the HFB, cell lines capable of optimal growth were selected. The Catharanthus roseus G. Don cell cultures that were used as the inoculum in the HFB were generated from stem and leaf callus. Fresh cells (10 g) were maintained in 100 mL of MS medium (Linsmaier, E. M., and Skoog, F., Physiol Plant 18:100-127 (1962)) supplemented with α-naphthalene acetic acid (1 mg/L), indole acetic acid (1 mg/L), kinetin (0.5 mg/L) and sucrose (40 g/L) in a 250-ml flask on a rotary shaker (120 RPM) at 25° C. in the dark.

To establish cell lines capable of optimal growth, cells were selected from shake-flask cultures called compact callus clusters measuring 5 to 8 mm in diameter showing some tissue differentiation. The compact callus clusters were then maintained in MS medium containing 2,4-Dichlorophenoxyacetic acid (2,4-D) (1 mg/L), which resulted in the high yielding PW-1 cell line. The PW-1 cell line was maintained in MS medium containing α-naphthalene acetic acid (1 mg/L), indole acetic acid (1 mg/L), kinetin (0.1 mg/L) and sucrose (40 g/L) at 25° C. in the dark. A second batch of Periwinkle suspensions (PW-2) was developed from calli that were cultured at 25° C. over a 16 hour photoperiod using cool-white fluorescent lighting (4-6 W/m²). After two weeks, PW-1 and PW-2 cells were washed with an alkaloid production medium, consisting of MS medium supplemented with indole acetic acid (1 mg/L), 6-benzylaminopurine (0.25 mg.L) and sucrose (40 g/L), and inoculated with 20 g inoculum/L into either a 250 mL flask (control) or into the 150 mL HFB bioreactor.

The HFB culture media was inoculated, through the sample port with the perfusion port open to allow air to escape, with Periwinkle medium by using a 60 cc glass syringe that had been sterilized. (All work with the HFB and cell cultures was performed using aseptic conditions and techniques inside a Laminar Flow hood that was cleaned with 70% ethanol.) This was done to acclimate the medium, test for leaks, and for contamination, while spinning the bioreactor at 25 RPM inside an incubator for 24 hours. After the 24 hour period, the reactor was drained of 20 mL of medium, and replaced with 20 mL (5 gm) of Periwinkle cell suspensions. Air bubbles were then pulled out through the sample port to make the HFB air-tight. The HFB bioreactor was operated at 25° C. in darkness. The cells slowly began to form tissue constructs at 25 RPM in 24 hours.

The results show that the PW-1 cells maintained in the dark had a creamy yellow and green appearance and lacked the gray turbid appearance of PW-2 cell cultures. Increased photoperiods and differences in light intensity during cell line development seem to have altered cells. PW-2 cell lines showed altered differences to increasing photoperiod and light intensity. Table 1 shows maximum specific growth rate (μ) and biomass doubling time (Td) for PW-1 and PW-2 shake-flask and HFB cultures. Specific growth rate measures cell mass concentration in grams/L and doubling time is measured in days.

TABLE 1
Specific Growth rates and Doubling Times of PW-1 and PW-2 cell
suspensions cultured in shake-flask vs. HFB conditions

| Culture Conditions | Specific Growth rate (μ) | Doubling time (Td) |
|---|---|---|
| Shake-flask (PW-1) | 0.13 | 8.0 |
| Shake-flask (PW-2) | 0.10 | 10.0 |
| HFB (PW-1) | 0.25 | 3.0 |
| HFB (PW-2) | 0.18 | 5.0 |

The lag phase typically observed in plant suspensions after inoculation in a conventional bioreactor was not readily apparent in microgravity conditions. The exponential phase for cell suspensions appears to begin almost immediately and lasted for 11-15 days in a HFB run that lasted 20 days. PW-1 cell cultures contained light green cells that grew rapidly and a cell biomass that increased to five times that of the inoculum biomass during the two weeks of HFB culture. This fast growth rate and biomass accumulation resulted in enough cell material for alkaloid production during the induction process.

The results show that the PW-1 cell cultures are superior to the PW-2 cell cultures with higher specific growth rates and lower doubling times as evidenced in both the shake-flask and HFB incubation experiments. Furthermore, the results show that increasing exposure to light during plant cell development from calli negatively influences the growth rates and doubling times of the resulting plant cell cultures.

### Example 3

### Osmotic Induction of Periwinkle Alkaloid Production

For the osmotic shock treatment of Periwinkle cells, 5%, 7%, 10% and 15% (w/v) mannitol was prepared in the growth medium. All of the mannitol preparations were adjusted to pH 5.8 before being autoclaved. Seven day-old PW-1 cell cultures were allowed to settle down, and 100 mL of spent medium was removed and replaced with 100 mL of the prepared media containing different concentrations of mannitol. Seven day-old three-dimensional tissues cultured in the HFB were treated by addition of varying mannitol concentrations. The control cell suspensions received the same volume of maintenance medium only. Alkaloid determination was carried out with PW-1 cells due to their faster doubling rate and their ability to withstand induction treatment without significant cell death. PW-1 cells were collected at intervals of 4 days within a 20 day culture cycle. Table 2 shows significant production of alkaloids (ajmalicine and catharanthine and serpentine) by osmotically challenging cells with 10% (−2.0 MPa) mannitol treatment. The results show that ten percent mannitol appears to be ideal for improved alkaloid yield compared to other concentrations tested (data not shown). The majority of the alkaloids were released into the medium with very little cell death. Serpentine production was small compared to ajmalicine and catharanthine production. Furthermore, about a ten-fold increase of catharathine and serpentine alkaloids was observed in HFB plant cell cultures compared to plant cells cultured in shake-flasks. Therefore, the natural production levels of alkaloids is increased by growing plant cells in the HFB and this alkaloid production level is further enhanced with the addition of mannitol to the media.

TABLE 2
Alkaloid production in PW-1 cell suspensions subjected to
chemical induction treatment.

| | Ajmalicine | | Catharanthine | | Serpentine | |
|---|---|---|---|---|---|---|
| Culture | (mg/g dry wt) | | (mg/g dry wt) | | (mg/g dry wt) | |
| time (days) | Flask | HFB | Flask | HFB | Flask | HFB |
| 0 | 0.10 | 0.12 | 0.07 | 0.10 | 0.04 | 0.08 |
| 4 | 0.35 | 1.2 | 0.20 | 0.85 | 0.10 | 0.55 |
| 8 | 0.85 | 2.0 | 0.50 | 1.5 | 0.39 | 1.25 |
| 12 | 1.25 | 3.5 | 0.85 | 2.12 | 0.49 | 1.97 |
| 16 | 1.80 | 4.6 | 0.98 | 3.78 | 0.70 | 2.25 |
| 20 | 1.35 | 5.0 | 0.75 | 4.10 | 0.56 | 2.08 |
| Control | 0.06 | 0.07 | 0.05 | 0.58 | 0.039 | 0.45 |

### Example 4

### Combined Induction by Abiotic and Biotic Inducing Agents on Periwinkle Alkaloid Production

Catharanthus roseus cell cultures were also treated with a combination of chemical and fungal inducing agents. The cell cultures significantly accumulated indole alkaloids as a result of this combination induction approach. A synergistic effect on alkaloid accumulation was observed in Catharanthus roseus cell cultures when treated with a combination of Aspergillum niger mycelial extract combined with PVP40 (Polyvinylpyrrolidone, MW: 40,000).

Aspergillum niger was grown in liquid potato dextrose medium for 7 days. Mycelia were collected by filtration and washed twice with deionized water, then homogenized in sodium acetate buffer (0.1 M, pH 5.8). The mycelium extract was autoclave sterilized. Catharanthus roseus cell cultures were induced with a 5% preparation of Aspergillum niger. One milliliter of this mycelium extract was added to the cultures, and samples were taken up to 80 hours after initial induction to determine alkaloid production. Two percent PVP40 (w/v) was dissolved in distilled water and filter-sterilized and used as the chemical inducing agent. Seven-day-old PW-1 cell suspensions either grown in shake-flasks or grown in the HFB and were treated by the addition of a combination of fungal (mycelium extract) and chemical (PVP) inducing agents.

TABLE 3
Alkaloid production in PW-1 cell suspensions subjected to
combined fungal and chemical induction treatment.

| | Ajmalicine | | Catharanthine | | Serpentine | |
|---|---|---|---|---|---|---|
| Culture | (mg/g dry wt) | | (mg/g dry wt) | | (mg/g dry wt) | |
| time (days) | Flask | HFB | Flask | HFB | Flask | HFB |
| 0 | 0.14 | 0.25 | 0.09 | 0.10 | 0.06 | 0.12 |
| 4 | 0.55 | 1.29 | 0.68 | 1.44 | 0.40 | 1.05 |
| 8 | 0.95 | 3.35 | 1.12 | 2.56 | 0.57 | 1.34 |
| 12 | 1.63 | 4.58 | 1.48 | 3.82 | 0.96 | 1.92 |
| 16 | 1.96 | 5.65 | 2.98 | 4.38 | 1.70 | 2.85 |
| 20 | 2.45 | 6.68 | 3.15 | 6.10 | 1.96 | 3.58 |

The results show that Catharanthus roseus cells grown in a microgravity environment showed higher accumulation of alkaloids with an osmotic stress agent and a combined induction treatment of fungal mycelium and a chemical. In the control cell culture and other non-induced cell cultures, total alkaloid production was extremely low. Microgravity conditions greatly facilitated the three-dimensional cell growth of Catharanthus roseus and influenced the increase in alkaloid production. These alkaloid increases are significant compared to shake-flask cell cultures at 1 G.

Alkaloid extraction and determination was carried out according to Shanks, et al. (1998) and Gupta et al (2001). (See e.g., Shanks, J. V., et al., Biotechnol. Bioeng. 58:333-338 (1998); Gupta, M. M., et al., Journal of chromatographic science, 43:450-453 (2005).) Using this method, alkaloids can be extracted into the ethyl acetate phase, and subsequently concentrated under vacuum. The alkaloids in the culture medium were extracted three times into the ethyl acetate phase and subsequently concentrated. The extracted alkaloid residues were dissolved in methanol and analyzed by HPLC.

Reverse phase liquid chromatography was performed isocratically with a mobile phase composed of acetonitrile:0.1 M phosphate buffer containing 0.5% glacial acetic acid (21:79, v/v; pH 3.5) with a flow rate of 1.2 mL/min and UV detection at 254 nm. HPLC peak purity and homogeneity of plant extract compounds was monitored using a photodiode-array detector. Compound identification was based on a comparison of peak retention time and UV spectra with ajmalicine, catharanthine (Fluka, St. Louis, Mo.), and serpentine standards (Research Plus, Bayonne, N.J.). Compound quantification was performed on chromatograms extracted at 254 and 329 nm.

### Example 5

### Microgravity Does Not Inhibit the Photosynthetic Apparatus

Plants have been proposed as the basis for a biological life support system that could be used alone or in concert with physical chemical life support systems to provide food, drugs and atmospheric purification on long-duration space flight missions. Successful development of the photosynthetic apparatus of plants during space flight is of paramount importance for such a scheme. However, to date, information about development of leaves and their photosynthetic performance in a microgravity environment has been scarce. The confocal microscopy observations of plant cells is centered on chloroplast structure, since this organelle is most quick to show disruptions in response to stress. There are frequent references in the space flight literature describing chloroplast disruptions.

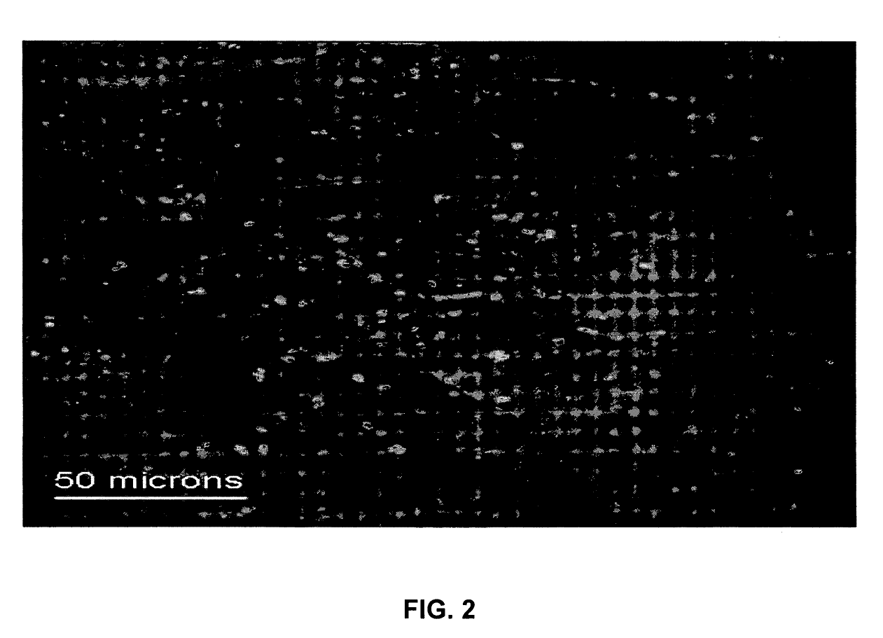

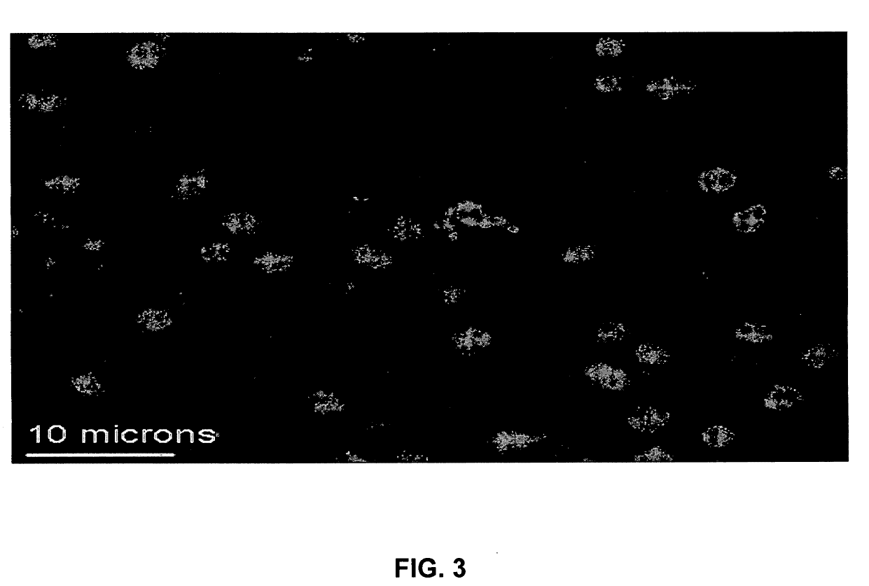

Confocal laser microscopy observations on Periwinkle cells subjected to microgravity in the HFB showed no disturbances in chloroplast structure compared to control cell cultures. Photosynthetic capacities of chloroplasts in Periwinkle cells appear to be normal as evidenced by oxygen bubble formation leading to the conclusion that microgravity does not inhibit the photosynthetic apparatus (FIG. 2). The integrity of the plastid membrane appears normal even at the end of four weeks of culture in the HFB. The only alteration that was observed in chloroplast structures was the slight swelling of chloroplasts at the end of a 7-10 day HFB culture (FIG. 3).

### Example 6

### Microgravity Promotes Three-Dimensional Tissue Formation

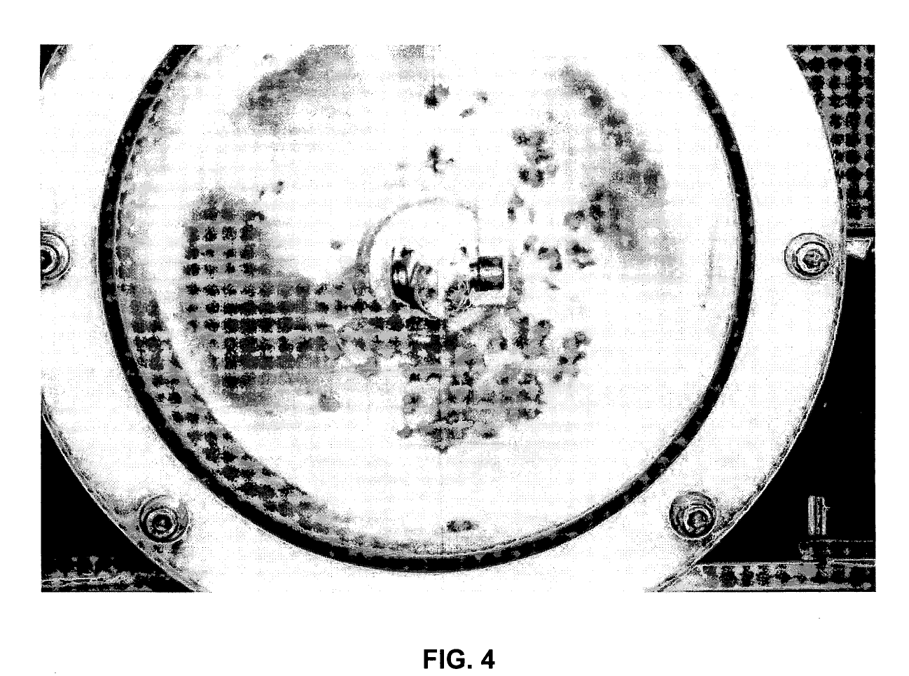

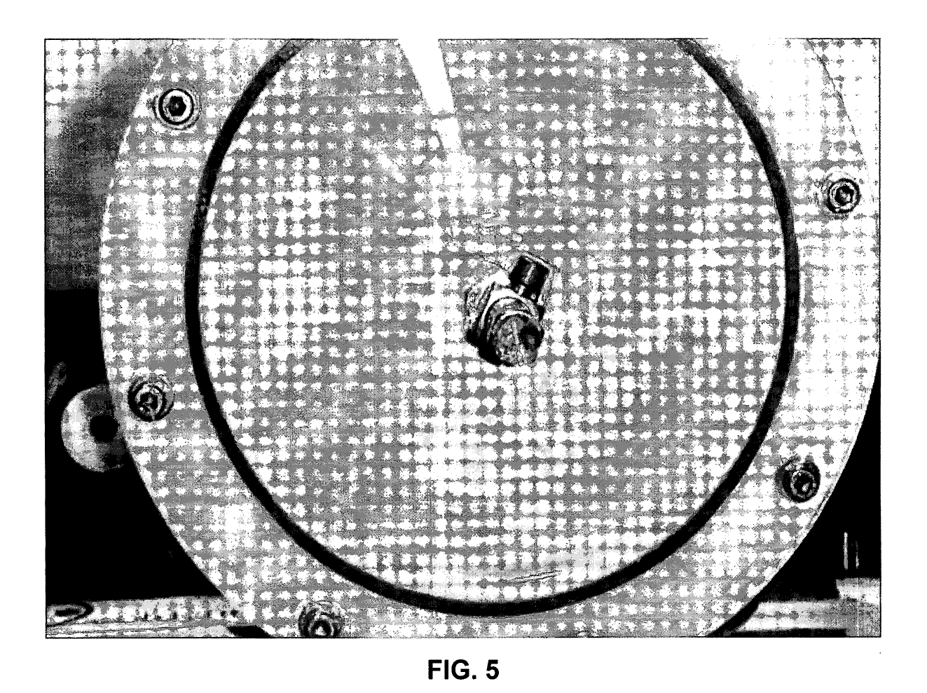

Plant cells cultured in a low shear microgravity environment of HFB can grow and differentiate to form three-dimensional cell tissues (FIGS. 4-5). It has been observed that at the end of 3 days, cultures grown in the HFB promote cell-cell interaction. In conventional tissue culture reactors such as Celligen-Plus, impellers create large shear forces to maintain cells in suspension, the cells generally slide past one another and detach from the tissue. Simulated microgravity allows cells to orient in three dimensions and to grow, differentiate and associate in a low shear environment. Plasmodesmata formation in the three-dimensional cell tissues was observed. Communication between plant cells largely occurs via intercellular connections, the plasmodesmata. Fine strands of cytoplasm, called plasmodesmata, extend through pores in the cell wall connecting the cytoplasm of each cell with that of its neighbors.

The results also show that culture conditions in the HFB provide an excellent in vitro system for studying the microenvironmental cues especially intercellular communication on tissue-specific cell assembly, differentiation and function. Photomixotrophic Periwinkle (Catharanthus roseus) cells cultured in the HFB for 7 days can assemble into macroscopic tissues several millimeters in size, devoid of necrotic cores. By 24 hours, cells were forming three-dimensional tissues. The fresh weight and dry weight of Periwinkle cell suspensions cultured in the HFB were monitored. The exponential growth phase appears to begin almost immediately and lasted throughout the seven-day culture period. The low-shear, simulated microgravity environment of the HFB helped in maintaining the survival of the three-dimensional Periwinkle tissues and Periwinkle (Catharanthus roseus) cells.

### Example 7

### Microgravity Influences the Abundance and Organization of Actin in Plant Cells

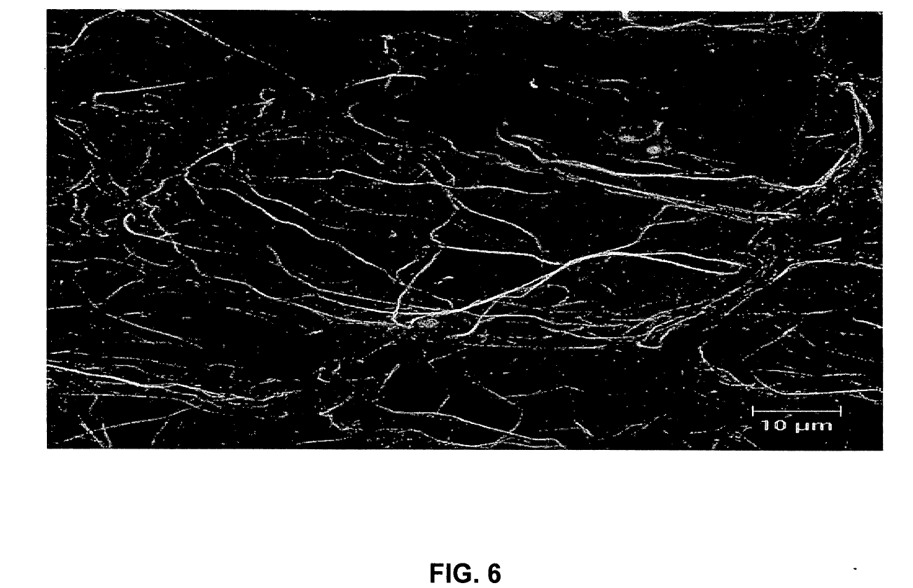

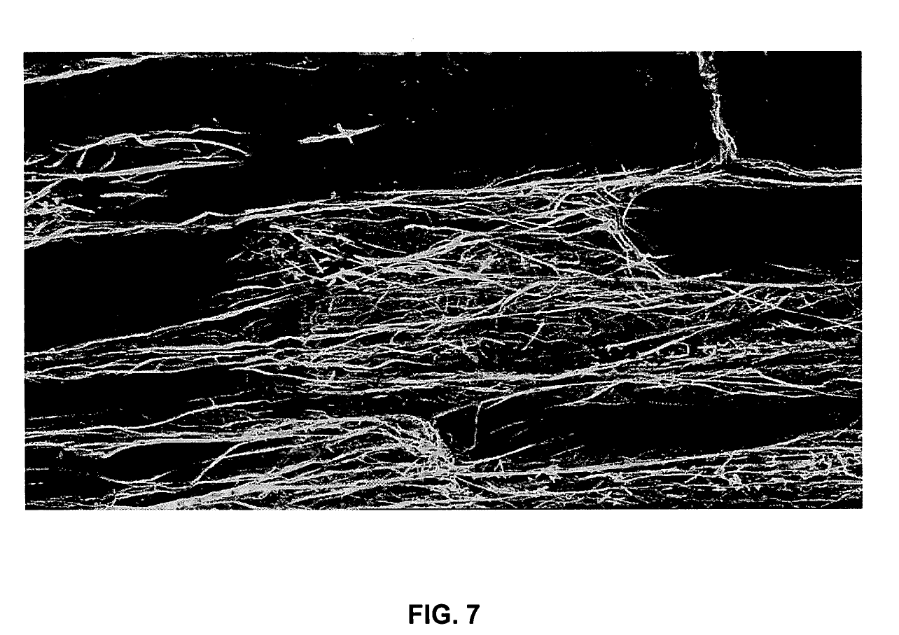

The plant actin cytoskeleton is characterized by high diversity with regard to gene families, isoforms, and degree of polymerization. In addition to the most abundant F-actin assemblies like filaments and their bundles, G-actin obviously assembles in the form of actin oligomers composed of a few actin molecules, which can be extensively cross-linked into complex dynamic meshworks. It was observed that the density of F-actin is affected by microgravity. Cells harvested at the end of a 7-day culture showed reduced density in F-actin compared to shake-flask control cells (FIGS. 6-7). It is possible that the actin cytoskeleton reorganizes and degrades following exposure to altered environmental condition of microgravity.

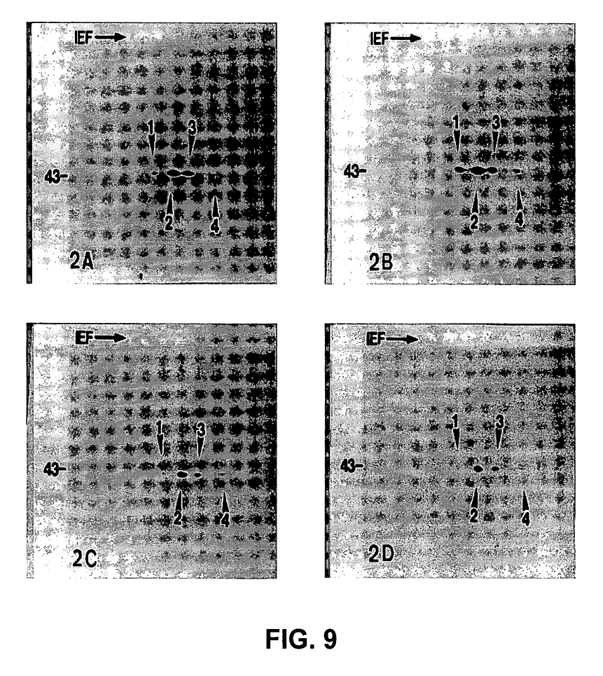

The pulling and/or pushing forces of the cytosol on the cell walls are detected within the plant cell and cell growth is adjusted accordingly. Actin-based microfilaments and proteins are integral components of the cellular cytoskeleton and are heavily influenced by gravitational forces. Periwinkle cells have four actin isoforms, which are constitutive polypeptides, and show a distinct distribution within the specific cellular compartments: two isoforms (pI 5.9 and 6.0) were found in plasma membrane and tonoplast preparations, whereas the pI 5.95 and 6.05 polypeptides were present in the soluble fraction. Immunoblot analysis of actin isoforms at the end of 2 days of HFB culture show a slight increase of the four major isoforms and a decrease during the microgravity phase ending with 5 and 7 days. At the end of the microgravity phase, only the spots corresponding to pI 6.0 and pI 5.95 were still visible compared to shake-flask control cells. The physiological meaning of this rapid decrease in actin isoforms remains unclear. One can speculate that a segment of the isoform population loses function under microgravity and undergoes degradation. More likely, actin isoform decline is a result of stress-induced proteolysis. It has been shown that the cellular organization is also disturbed in gravity-insensitive cells. Those disturbances are believed to cause stress reactions influencing the protein metabolism as reflected by the microgravity-affected ubiquitin pools. These results provide strong evidence that microgravity has a direct positive influence on protein metabolism in Periwinkle plant cells (FIG. 9).

### Example 8

### Microgravity Influences Protein Metabolism in Plant Cells

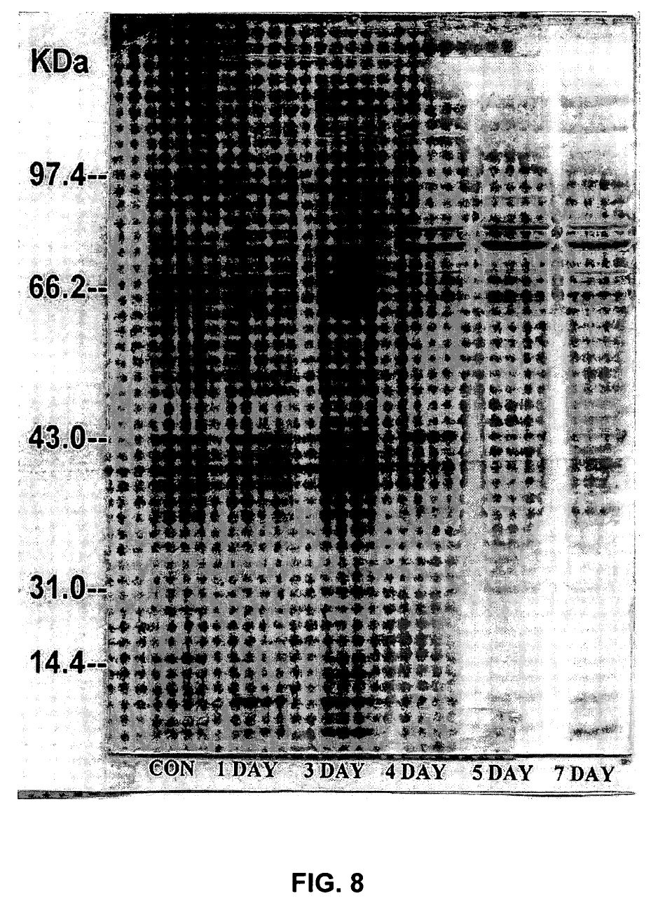

Periwinkle plant cells from cultures under both control and microgravity conditions were lysed and the total protein was separated by 1-D gel electrophoresis. Using one-dimensional electrophoresis and fluorography of de novo synthesis proteins, it was possible to follow changes in the pattern of protein synthesis in Periwinkle cells subjected to microgravity. Of the newly-synthesized proteins visualized by fluorography, a new 85 kD protein showed strong enhanced expression in cells subjected to 2, 3, 5 and 7 days of microgravity conditions and a 43 kD protein showed transiently increased expression in cells (FIG. 8). The results indicated that microgravity enhances protein expression.

### Example 9

### Monitoring Secondary Metabolism in Periwinkle HFB Cell Suspensions

Two precursor enzymes that are expressed throughout the indole alkaloid pathway for secondary metabolism with Periwinkle plant cells are tryptophan decarboxylase (TDC) and strictosidine synthase (STR). TDC and STR precursor enzyme expression is important for the production of ajmalicine and serpentine, or vinblastine and vincristine. Therefore, TDC and STR gene expression in Periwinkle cells was monitored, using Northern Blots. After a control was established, induction was carried out at 2, 4, 8, 12 and 24 hours. A transient increase in both genes was observed to indicate that this pathway was activated (results not shown).

### Example 10

### Isoflavonoid Production in Sandalwood HFB Cell Suspensions

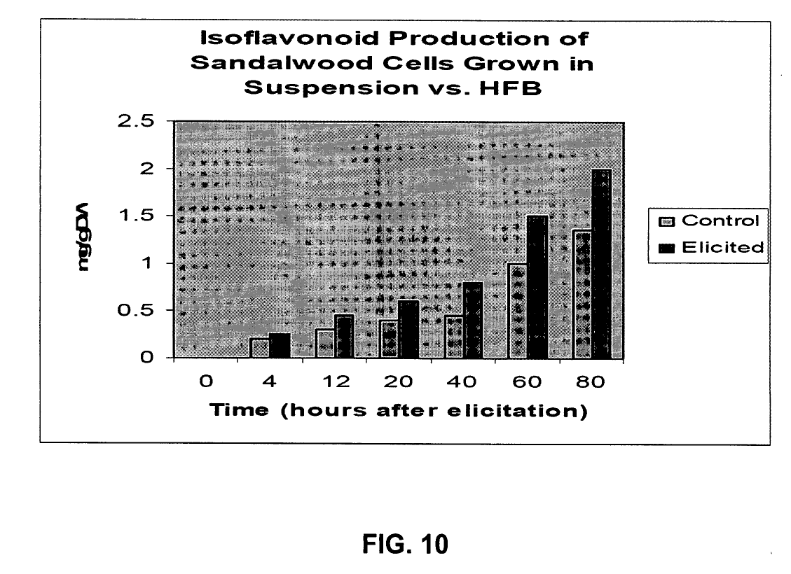

Sandalwood (Santalum album L.) plant cell cultures were established by methods similar to those used in Example 2 in establishing Periwinkle cell cultures. After being inoculated into the HFB, the Sandalwood cultures were induced with mannitol. The results showed a two-fold increase in isoflavonoids, the secondary metabolite produced in Sandalwood cells, was observed in the HFB cell tissues compared to the shake-flask cell suspensions (FIG. 10).

### Example 11

### Kinetics of Growth, Uptake of Macronutrients and Accumulation of Indole Alkaloids in Periwinkle HFB Cell Suspensions

The kinetics of growth, the uptake of macronutrients, and the accumulation of indole alkaloids from Periwinkle (Catharanthus roseus) cells were investigated. The doubling time [dry-weight (DW) basis] of Catharanthus roseus cells in B5/2 nutrients supplemented with 3% sucrose was 3.0 days. NH₄⁺, NO₃⁻ and Pi were depleted sequentially from culture medium by the cells, while the concentration of sugars remained same. Medium pH decreased to 4.8 in early exponential phase of Catharanthus roseus culture growth from the initially adjusted pH value of 5.7, and increased subsequently to a maximum of 7.7 in late exponential phase of growth coincident with the maximum of fresh weight (FW)/DW ratio, before decreasing to pH 4.8. This drop in pH was attributed to the presence of organic acids, pyruvate, lactate, and succinate in the late phase of exponential growth, possibly resulting in the late-culture pH decrease. Accumulation of an alkaloid (tabersonine) was distinctly growth-associated with maximum specific and total yields of 1.0 mg/g DW and 3.0 mg/L, respectively, in late-exponential phase of growth. Serpentine accumulation was non-growth associated with increasing specific and total levels in stationary growth phase: 1.2 mg/g DW and 8.0 mg/L, respectively.

Cell growth rates and alkaloid production are also dependent on the culture temperature. Catharanthus roseus cell cultures maintained at 25° C. were grown in MS medium supplemented with 2% sucrose at various temperatures from 10° C. to 45° C. Plant cell growth rates were maximal at 35° C. but declined rapidly above 35° C. and below 25° C. Maximum serpentine yields were obtained between 20° C. and 25° C. Serpentine yields fell sharply when the cell cultures were maintained at temperatures above 25° C. and below 20° C. Maximum ajmalicine yields were obtained when the cell cultures were maintained at 20° C. The variable serpentine/ajmalicine ratio at different growth temperatures suggests that lower temperatures may favour ajmalicine accumulation. Both the growth rate and the rate of alkaloid accumulation at 25° C. were sensitive to small changes in average culture temperature.

### Example 12

### Methods for Microsphere Production and Microencapsulation of Cells

Microencapsulation of cells allows for high density cultures to be protected from the shear damage in flow or stirred systems. They also provide suitable surfaces for anchorage-dependent species of plant cells when microcarrier beads and coencapsulated with the cells. Calcium-alginate/chitosan microspheres were prepared by the addition of droplets (ca. 0.1 mL in volume) of a slightly viscous solution of sodium alginate (2 wt %, Sigrna Aldrich) in aqueous NaCl (0.15 M) to an aqueous solution of chitosan (1 wt % Sigma Aldrich) containing CaCl₂ (50 mM) and 1 wt % acetic acid at pH 6.2-6.5. The droplets were added via a 0.4 mm diameter needle syringe and remained in the chitosan solution for 1 hour. Typically, a hundred microcapsules or so can be prepared in a single experiment.

To create the cell-alginate mixture, a 1 mL aliquot of soybean (Glycine max) cell suspension with a seeding density of 5×10⁷ cells/mL was added to 9 mL of a 2.2% (w/v) sodium alginate solution to yield a final microencapsulated cell seeding sample.

### Example 13

### Establishing Bacterial Cell HFB Cultures

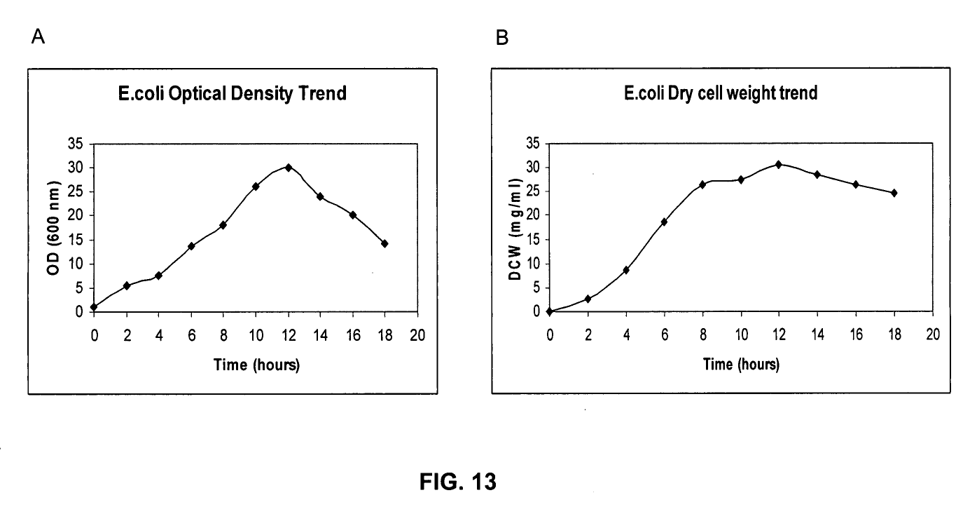

Bacterial cells can also be readily cultured in the HFB. Escherichia coli MC1061 cells, previously stored in a 10% (w/v) glycerol stock at −20° C., were initially grown at 37° C. in a LB broth stock solution for 24 hours and sub-cultured twice at 37° C. for 12 hours after transfer to culture media. The last sub-culture was centrifuged at 5000 g for 5 minutes. The cell mass was resuspended to the necessary optical density (OD) in fresh K12 nutrient medium. K12 medium consists of 2 g/L anhydrous potassium phosphate (monobasic), 3 g/L anyhdrous potassium phosphate (dibasic), 5 g/L anhydrous ammonium phosphate (dibasic), 5 g/L Tastone 900AG, 25 g/L glucose, 0.5 g/L magnesium sulfate heptahydrate, 1 mg/L thiamine and 0.5 mL/L of a K12 trace metal solution and adjusted to a pH of 7.5. The K12 trace metal solution consists of 5 g/L of sodium chloride, 1 g/L zinc sulfate heptahydrate, 4 g/L manganese chloride tetrahydrate, 4.75 g/L ferric chloride hexahydrate, 0.4 g/L cupric sulfate pentahydrate, 0.575 g/L boric acid, 0.5 g/L sodium molybdate dihydrate and 12.5 mL/L of 6N sulfuric acid. OD measured at 600 nm was 5.50 at the time of inoculation. Inoculum volume was 5% of the 160 mL working volume of the HFB. Growth was monitored using light scattering by measuring the OD values at 600 nm in quartz cuvettes with a 10-mm light path in a spectrophotometer. FIG. 13 shows the increase of OD at 600 nm measured for the bacterial cell culture over a 20 hour incubation period. The results show that biomass within the bioreactor increases in the first 10 hours of incubation and then slowly decreases in the following 10 hours of incubation.

### Example 14

### Establishing Fungal Cell HFB Cultures

Fungal cell cultures can also be established within the HFB. Saccharyomyces cerevisiae yeast cells are grown overnight in YPD medium containing 1% yeast extract, 2% polypeptone and 2% glucose at 30° C. in shake-flasks and are inoculated into freshly prepared YPD medium to give an initial cell density of approximately 10⁶ cell/mL. Samples of cell culture are withdrawn from the HFB at discrete time intervals to measure the cell density (OD measured at 610 nm). Cell culture samples are also measured for colony forming units (cfu) by plating appropriately diluted samples on YPD agar plates and incubating these plates at 30° C. A yeast cell suspension of 10⁶ cells/mL will give an OD value of approximately 0.1.

1. A method for continuous culture of plant cells comprising growing the cells in a hydrofocusing bioreactor (HFB) under conditions sufficient for growth.

2-27. (canceled)

28. A method for producing one or more bioactive compounds, comprising continuously culturing plant cells in a hydrofocusing bioreactor under conditions sufficient for production of one or more bioactive compounds by said plant cells, and isolating said bioactive compounds.

29-61. (canceled)

62. A method for assaying the presence of one or more bioactive plant compounds, comprising continuously culturing plant cells in a hydrofocusing bioreactor, whereby said plant cells produce the bioactive compounds.

63-91. (canceled)

92. A process for obtaining a tissue-like, three-dimensional plant cell construct in a hydrofocusing bioreactor, comprising filling the culture chamber of said hydrofocusing bioreactor with a medium and plant cells of one or more distinct types to establish a culturing environment within the culture chamber and continuously culturing the plant cells from at least about 3 days to about 35 days.

93-97. (canceled)

98. A tissue-like three-dimensional plant cell construct grown in a hydrofocusing bioreactor, wherein the tissue-like three-dimensional plant cell construct has a reorganized and degraded cytoskeleton and swollen chloroplasts.

99. A method for continuous culture of fungal cells comprising growing the cells in a hydrofocusing bioreactor (HFB) under conditions sufficient for growth.

100. A method for continuous culture of bacterial cells comprising growing the cells in a hydrofocusing bioreactor (HFB) under conditions sufficient for growth.
